(19) United States
(12) Patent Application Publication
(10) Pub. No.: US 2021/0145818 A1
(43) Pub. Date: May 20, 2021

(54) COMPOSITIONS INFUSED WITH NICOTINE COMPOUNDS AND METHODS OF USE THEREOF

(71) Applicant: POVIVA TEA, LLC, Reno, NV (US)

(72) Inventors: John Docherty, Port Perry (CA); Christopher Andrew Bunka, Kelowna (CA)

(21) Appl. No.: 17/047,473

(22) PCT Filed: Apr. 16, 2019
(86) PCT No.: PCT/IB2019/000505

§ 371 Date: Oct. 14, 2020

Related U.S. Application Data
(60) Provisional application No. 62/658,473, filed on Apr. 16, 2018.
(60) Provisional application No. 62/748,514, filed on Oct. 21, 2018.

(51) Int. Cl. A61K 31/465; A23L 33/105; A23L 33/12; A23L 33/155; A61K 45/06; A23F 3/14; A61K 9/19; A23L 27/00

(52) U.S. Cl. CPC A61K 31/465; A23L 33/105; A23L 33/12; A23L 27/84; A61K 45/06; A23F 3/14; A61K 9/19; A23L 33/155

(57) ABSTRACT

Aspects described herein relate to compositions, including edible compositions, infused with nicotine compounds and methods of use for the treatment of a nicotine-related disorders. More particularly, aspects described herein relate to compositions, including edible compositions, infused with nicotine compounds that provide enhanced bioavailability of the nicotine compounds in a subject, and that mask unpleasant tastes of the nicotine compounds.

### CROSS-REFERENCE TO RELATED APPLICATIONS

This application is a PCT International Application which claims the benefit of U.S. Provisional Application No. 62/658,473, filed Apr. 16, 2018; and U.S. Provisional Application No. 62/748,514, filed Oct. 21, 2018; each of which are incorporated herein by reference in their entireties.

### TECHNICAL FIELD

Aspects described herein relate to compositions, including edible compositions, infused with nicotine compounds and methods of use for the treatment of a nicotine-related disorders. More particularly, aspects described herein relate to compositions, including edible compositions, infused with nicotine compounds that provide enhanced bioavailability of the nicotine compounds in a subject, and that mask unpleasant tastes of the nicotine compounds.

### BACKGROUND

It is generally believed that regular cigarettes or cigars filled with tobacco can cause numerous health problems, such as cancer and heart disease. A major limiting factor in the successful use of nicotine replacement therapy for smoking cessation is the lack of an appropriate delivery system. When a person smokes a cigarette, the level of nicotine rises rapidly in the blood and in the brain, with an interval of just 10 to 20 seconds between taking a puff and the nicotine arriving in the brain. Presently marketed nicotine replacement products such as the transdermal nicotine patch and the nicotine chewing gum are not entirely satisfactory in that they do not provide the patient with the nicotine “buzz” associated with smoking a cigarette. This is because both the patch and the gum are slowly acting controlled release systems where only low nicotine plasma levels are obtained.

The nicotine chewing gum is a slow release preparation where the rate of release of nicotine will depend on the rate of chewing. It takes 20 to 30 min of vigorous chewing to release 95% of the nicotine content of the gum. Without chewing or if the gum is accidently swallowed negligible amounts of nicotine are released. The gum contains 2 or 4 mg of nicotine. A typical smoker needs about 15 pieces of gum a day. The gum has an unpleasant taste and may be irritating to the mouth and throat. Potential side effects are heartburn and hiccups. Tired and aching jaws may be experienced from intensive chewing and users rarely maintain blood nicotine concentrations above one third of their levels from smoking. Furthermore, the chewing gum is contraindicated in individuals with gastritis or active peptic ulcer disease and presents difficulties for those wearing dentures.

The nicotine patch, when placed on the skin, will give a steady release of nicotine over 24 hours and should be changed daily. With the patch in place it takes 3 to 4 hours to attain significant blood levels of nicotine. The continuous dosing provided by patches can disrupt the usual day/night variation in nicotine intake provided by smoking and can result in a total dose of nicotine per 24 hours exceeding the normal smoking dose. Moreover, it seems that if nicotine is given both night and day compared to only daytime, sleep disturbances and nightmares can result. Another potential side effect of the patch is skin irritation. A further disadvantage with the nicotine patch is that it is a passive system and for some individuals, a closer involvement with the treatment is to be preferred.

Some experts believe that electronic cigarettes (also known as e-cigarettes, e-cigs, and personal vaporizers) may be used as a potentially healthier alternative to smoking regular cigarettes (i.e., cigarettes that produce smoke by combustion of tobacco and/or other ingredients).

E-cigarettes are generally battery-powered devices, which produce an inhalable aerosol (referred to as “vapor”) by atomizing a liquid solution (“e-liquid” or “e-juice”), generally using a heating element such as a metal coil. E-liquid is typically composed of a mixture of one or more of propylene glycol (pg) and vegetable glycerin (vg), combined with flavorings, nicotine, or other psychoactive chemical ingredients. The vapor produced by e-cigarettes may include fewer carcinogens and other unhealthy chemicals, which may improve health outcomes for users of e-cigarettes as compared to users of traditional cigarette or tobacco products.

Some vaping devices include a tank, which may hold for example 1 mL, 2 mL, or 5 mL of e-liquid at a time. A wick draws the e-liquid into an atomizer, which heats it with a coil and produces vapor. Other vaping devices, known as “drippers” require the user to manually add a few drops of e-liquid directly to the coils of an atomizer. For both tank and dripper devices, frequent users must keep e-liquid on hand in order to refill the device. In addition to the inconvenience of storing and using multiple types of e-liquids, there are safety concerns as well. E-liquids that contain liquid nicotine are poisonous if ingested. Some users have noted the danger of having colorful flavored liquids, which may be attractive to children, containing concentrated nicotine that is in fact harmful if ingested.

In addition to the convenience and safety concerns, prior art e-liquid delivery systems have other drawbacks as well. For example, aerosolizing e-liquid by heating may cause degradation and pyrolysis of some chemical species residing in the mixture. Flavors and active ingredients may be lost or chemically altered, yielding a suboptimal vaping experience.

Therefore, there is a need for improved compositions and methods for the administration of nicotine compounds to subjects in need thereof.

### SUMMARY

To address the foregoing problems, in whole or in part, and/or other problems that may have been observed by persons skilled in the art, the present disclosure provides compositions and methods as described by way of example as set forth below.

An edible product infused with a nicotine compound is provided, comprising:
(a) a therapeutically effective amount of a nicotine compound;
(b) a bioavailability enhancing agent, wherein the bioavailability enhancing agent comprises an edible oil comprising long chain fatty acids and/or medium chain fatty acids and enhances the bioavailability of the nicotine compound; and
(c) an edible substrate. In some embodiments, the edible product is obtainable by the steps of:
(i) contacting the edible substrate with an oil comprising the nicotine compound and the bioavailability enhancing agent; and
(ii) dehydrating the edible substrate;
   thereby producing the edible product infused with a nicotine compound.

In some embodiments, the edible product is selected from the group consisting of a pill, tablet, lozenge, mini lozenge, capsule, caplet, pouch, gum, spray, food, and combinations thereof.

The edible product of claim 1, wherein the nicotine compound is selected from the group consisting of nicotine, free base nicotine, pharmacologically acceptable salts of nicotine, a nicotine complex, and polymer resins of nicotine.

The edible product of claim 4, wherein the salt of nicotine is an acid addition salt selected from the group consisting of nicotine hydrogen tartrate, nicotine bitartrate dihydrate, nicotine hydrochloride, nicotine dihydrochloride, nicotine sulfate, nicotine citrate, nicotine zinc chloride monohydrate, nicotine salicylate, nicotine oil, and nicotine complexed with cyclodextrin.

The edible product of claim 4, wherein the polymer resin is selected from the group consisting of nicotine polacrilex and nicotine resinate.

The edible product of claim 1, wherein the nicotine compound is selected from the group consisting of Nicotine, (s)-Nicotine, Nornicotine, (S)-Cotinine, B-Nicotyrine, (S)-Nicotene-N′-Oxide, Anabasine, Anatabine, Myosmine, B-Nornicotyrine, 4-(Methylamino)-1-(3-pyridyl)-1-butene (Metanicotine) cis or trans, N′-Methylanabasine, N′Methylanatabine, N′Methylmyosmine, 4-(Methylamino)-1-(3-pyridyl)-1-butanone (Pseudoxynicotine), and 2,3′-Bipyridyl.

The edible product of claim 1, wherein the nicotine compound is selected from the group consisting of nicotine bitartrate, lobeline, cytisine, nicotine polacrilex, nornicotine, nicotine 1-N-oxide, metanicotine, nicotine imine, nicotine N-glucuronide, N-methylnicotinium, N-n-decylnicotinium, 5′-cyanonicotine, 3,4-dihydrometanicotine, N′-methylnicotinium, N-octanoylnornicotine, 2,3,3a,4,5,9b-hexahydro-1-methyl-1H-pyrrolo(3,2-h)isoquinoline, 5-isothiocyanonicotine, 5-iodonicotine, 5′-hydroxycotinine-N-oxide, homoazanicotine, nicotine monomethiodide, N-4-azido-2-nitrophenylnornicotine, N-methylnornicotinium, nicotinium molybdophosphate resin, N-methyl-N′-oxonicotinium, N′-propylnornicotine, pseudooxynicotine, 4′-methylnicotine, 5-fluoronicotine, K(s-nic)5(Ga2(N,N′-bis-(2,3-dihydroxybenzoyl)-1,4-phenylenediamine)3), 5-methoxynicotine, 1-benzyl-4-phenylnicotinamidinium, 6-n-propylnicotine, SIB1663, 6-hydroxynicotine, N-methyl-nicotine, 6-(2-phenylethyl)nicotine, N′-formylnornicotine, N-n-octylnicotinium, N-(n-oct-3-enyl)nicotinium, N-(n-dec-9-enyl)nicotinium, 5′-acetoxy-N′-nitrosonornicotine, 4-hydroxynicotine, 4-(dimethylphenylsilyl)nicotine, N′-carbomethoxynornicotine, and N-methylnicoton.

The edible product of claim 1, wherein the nicotine compound is an agonist having selectivity to an α₇ nicotinic receptor subtype, wherein the agonist is selected from the group consisting of N-[(2S,3 S)-2-(pyridin-3-ylmethyl)-1-azabicyclo[2.2.2]oct-3-yl]-1-benzofuran-2-carboxamide, (5aS,8 S,10aR)-5a,6,9,10-Tetrahydro,7H,11H-8,10a-methanopyrido[2′,3′:5,6]pyrano[2,3-d]azepine, 1,4-Diazabicyclo[3.2.2]nonane-4-carboxylic acid, 4-bromophenyl ester, 3-[(3E)-3-[(2,4-dimethoxyphenyl)methylidene]-5,6-dihydro-4H-pyridin-2-yl]-pyridine, 2-methyl-5-(6-phenyl-pyridazin-3-yl)-octahydro-pyrrolo[3,4-c]pyrrole, (5S)-spiro[1,3-oxazolidine-5,8′-1-azabicyclo[2.2.2]octane]-2-one, N-[(3R)-1-azabicyclo[2.2.2]oct-3-yl]-4-chlorobenzamide, 5-morpholin-4-yl-pentanoic acid (4-pyridin-3-yl-phenyl)-amide, EVP-6124, EVP-4473, TC-6987, and MEM3454.

The edible product of claim 1, wherein the nicotine compound is an agonist having selectivity to an α₄β₂ nicotinic receptor subtype, wherein the agonist is selected from the group consisting of 7,8,9,10-tetrahydro-6,10-methano-6H-pyrazino(2,3-h)(3) benzazepine, (2S,4E)-5-(5-isopropoxypyridin-3-yl)-N-methylpent-4-en-2-amine, [3-(2(S))-azetidinylmethoxy)pyridine] dihydrochloride, (5aS,8S,10aR)-5a,6,9,10-Tetrahydro,7H,11H-8,10a-methanopyrido [2′,3′:5,6]pyrano[2,3-d]azepine, A-969933, S35836-1, S35678-1, and 3-(5,6-Dichloro-pyridin-3-yl)-1S,5S-3,6-diazabicyclo[3.2.0]heptane.

In some embodiments, the edible substrate is selected from the group consisting of inulin, starch, modified starches, xanthan gum, carboxymethyl cellulose, methyl cellulose, hydroxypropylmethyl cellulose, konjac, chitosan, tragacanth, karaya, ghatti, larch, carageenan, alginate, chemically modified alginate, agar, guar, locust bean, psyllium, tara, gellan, curdlan, pullan, gum arabic, gelatin, pectin, and combinations thereof.

In some embodiments, the edible product further comprises a flavoring agent selected from the group consisting of vanilla, vanillin, ethyl vanillin, orange oil, fruit and berry type flavorants, Dramboui, bourbon, scotch, whiskey, spearmint, lavender, cinnamon, chai, cardamon, apium graveolents, clove, cascarilla, nutmeg, sandalwood, bergamot, geranium, honey essence, rose oil, lemon oil, Japanese mint, cassia, caraway, cognac, jasmin, chamomile, menthol, ylang ylang, sage, fennel, pimenta, ginger, anise, chai, coriander, coffee, peppermint, wintergreen, mint oils from a species of the genus Mentha, and combinations thereof.

In some embodiments, the edible product further comprises an additive selected from the group consisting of a non-nicotine alkaloid, a mineral, a vitamin, a dietary supplement, a dietary mineral, a nutraceutical, an energizing agent, a soothing agent, a coloring agent, an amino acid, a chemsthetic agent, an antioxidant, a food grade emulsifier, a pH modifier, a botanical, a teeth whitening agent, a therapeutic agent, a sweetener, a flavorant, and combinations thereof.

In some embodiments, the bioavailability of the nicotine compound in a subject is at least 2 times, 5 times, or 10 times greater than the bioavailability of the nicotine compound in the subject in the absence of the edible oil comprising long chain fatty acids and/or medium chain fatty acids. In some embodiments, the edible oil comprising long chain fatty acids and/or medium chain fatty acids is substantially free of omega-6 fatty acids. In some embodiments, the long chain fatty acids and/or medium chain fatty acids are selected from the group consisting of oleic acid, undecanoic acid, valeric acid, heptanoic acid, pelargonic acid, capric acid, lauric acid, and eicosapentaenoic acid.

In some embodiments, the edible product further comprises a secondary active agent, wherein the secondary active agent is a lipophilic active agent selected from the group consisting of: cannabinoids, terpenes and terpenoids, non-steroidal anti-inflammatory drugs (NSAIDs), vitamins, nicotine or an analog thereof, phosphodiesterase 5 (PDE5) inhibitors, Maca extract, hormones, fentanyl or an analog thereof, buprenorphine or an analog thereof, scopolamine or an analog thereof, and antioxidants. In some embodiments, the cannabinoid is a psychoactive cannabinoid. In some embodiments, the cannabinoid is a non-psychoactive cannabinoid. In some embodiments, the NSAID is acetylsalicylic acid, ibuprophen, acetaminophen, diclofenac, indomethacin, piroxicam, or a COX inhibitor. In some embodiments, the vitamin is vitamin A, D, E, or K. In some embodiments, the PDE5 inhibitor is avanafil, lodenafil, mirodenafil, sildenafil, tadalafil, vardenafil, udenafil, acetildenafil, thiome-thisosildenafil, or analogs thereof. In some embodiments, the hormone is an estrogen, an anti-estrogen, an androgen, an anti-androgen, or a progestin. In some embodiments, the antioxidant is astaxanthin, Superoxide Dismusase, beta-carotene, selenium, lycopene, lutein, Coenzyme Q10, phytic acid, flavonoids, a polyphenol, a substituted 1,2-dihydroquinoline, ascorbic acid and its salts, ascorbyl palmitate, ascorbyl stearate, anoxomer, N-acetylcysteine, benzyl isothiocyanate, o-, m- or p-amino benzoic acid (o is anthranilic acid, p is PABA), butylated hydroxyanisole (BHA), butylated hydroxytoluene (BHT), caffeic acid, canthaxantin, alpha-carotene, beta-carotene, beta-caraotene, beta-apo-carotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids, flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, ice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., lonox 100), 2,4-(tris-3′,5′-bi-tert-butyl-4′-hydroxybenzyl)-mesitylene (i.e., lonox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivates, vitamin Q10, wheat germ oil, zeaxanthin, or combinations thereof.

In some embodiments, the edible product is a food product, wherein the edible substrate is selected from the group consisting of tea leaves, coffee beans, cocoa powder, meats, fish, fruits, vegetables, dairy products, legumes, pastas, breads, grains, seeds, nuts, spices, and herbs. In some embodiments, the bioavailability enhancing agent is a protective colloid, an edible oil or fat, and a lipophilic active agent taste masking agent. In some embodiments, the bioavailability enhancing agent that is a protective colloid, an edible oil or fat, and a lipophilic active agent taste masking agent is nonfat dry milk. In some embodiments, the edible product is lyophilized.

In some embodiments, a process for making an edible product infused with a nicotine compound is provided, comprising:
(a) providing a therapeutically effective amount of a nicotine compound;
(b) providing a bioavailability enhancing agent, wherein the bioavailability enhancing agent comprises an edible oil comprising long chain fatty acids and/or medium chain fatty acids and enhances the bioavailability of the nicotine compound;
(c) providing an edible substrate;
(d) contacting the edible substrate with an oil comprising the nicotine compound and the bioavailability enhancing agent; and
(e) dehydrating the edible substrate;
   thereby producing the edible product infused with a nicotine compound.

In some embodiments, a beverage product infused with a nicotine compound is provided obtainable by the steps of:
(i) providing the edible product infused with a nicotine compound as described above, wherein the edible product infused with a nicotine compound is tea leaves, coffee beans, or cocoa powder infused with a nicotine compound; and
(ii) steeping the tea leaves, coffee beans, or cocoa powder infused with a nicotine compound in a liquid;
   thereby producing the beverage product infused with the nicotine compound. In some embodiments, the beverage product infused with a nicotine compound is obtainable by the steps of:
(i) providing the edible product infused with a nicotine compound as described above, wherein the edible product infused with a nicotine compound is tea leaves, coffee beans, or cocoa powder infused with a nicotine compound; and
(ii) steeping the tea leaves, coffee beans, or cocoa powder infused with a nicotine compound in a liquid;
   thereby producing the beverage product infused with the nicotine compound.

In some embodiments, a method of treating a nicotine-related disorder is provided comprising administering the edible product infused with a nicotine compound or the beverage product infused with a nicotine compound to a subject in need thereof, and wherein the nicotine-related disorder is selected from the group consisting of tobacco dependence/addiction, Parkinson's disease, ulcerative colitis, Alzheimer's disease, schizophrenia, Attention Deficit Hyperactivity Disorder (ADHD), Tourette's syndrome, ulcerous colitis, and post-smoking-cessation weight control.

In some embodiments, a method of enhancing the bioavailability of a nicotine compound is provided, comprising heating the edible product infused with a nicotine compound or the beverage product infused with a nicotine compound to a temperature that is greater than or equal to human body temperature. In some embodiments, the method of enhancing the bioavailability of a lipophilic active agent comprises oral administration of the edible product infused with a nicotine compound or the beverage product infused with a nicotine compound to a human subject.

In some embodiments, a kit is provided comprising the edible product or the beverage product infused with a nicotine compound and instructions for use thereof.

Other compositions, methods, features, and advantages of the invention will be or will become apparent to one with skill in the art upon examination of the following figures and detailed description. It is intended that all such additional compositions, methods, features, and advantages be included within this description, be within the scope of the invention, and be protected by the accompanying claims.

### BRIEF DESCRIPTION OF THE DRAWINGS

The invention can be better understood by referring to the following figures. The components in the figures are not necessarily to scale, emphasis instead being placed upon illustrating the principles of the invention.

FIG. 1 shows results from Example 5 comparing nicotine concentrations in various tissues following administration of DEHYDRATECH™ and control compositions in rats.

FIG. 2 shows results from Example 6 showing improvement in peak nicotine blood levels following administration of DEHYDRATECH™ and control compositions in rats.

FIG. 3 shows results from Example 6 comparing nicotine concentrations in various tissues following administration of DEHYDRATECH™ and control compositions in rats.

FIG. 4 shows results from Examples 5 and 6 comparing improvements in maximum brain concentration, time to C max, and total quantity in brain tissue following administration of DEHYDRATECH™ and control compositions in rats.

### DETAILED DESCRIPTION

The presently disclosed subject matter now will be described more fully hereinafter. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

In some embodiments, the compositions or methods comprise the specified components or steps. In some embodiments, the compositions or methods consist of the specified components or steps. In other embodiments, the compositions or methods consist essentially of the specified components or steps. As used herein, “consists essentially of” the specified components or steps means that the composition includes at least the specified components or steps, and may also include other components or steps that do not materially affect the basic and novel characteristics of the invention.

The present invention is directed to compositions, including edible compositions, infused with nicotine compounds that provide enhanced bioavailability in a subject, particularly wherein the unpleasant taste of the nicotine compound is masked. Processes for making the compositions, including edible compositions, are also provided, as well as methods for treating nicotine-related disorders comprising administering any of the compositions disclosed herein to a subject in need thereof. Methods of enhancing the bioavailability of a nicotine compound are also provided, comprising oral administration of the edible product infused with a nicotine compound or the beverage product infused with a nicotine compound to a human subject. Kits are also provided comprising the edible product or the beverage product infused with a nicotine compound and instructions for use thereof.

### I. Compositions

An edible product infused with a nicotine compound is provided, comprising:
(a) a therapeutically effective amount of a nicotine compound;
(b) a bioavailability enhancing agent, wherein the bioavailability enhancing agent comprises an edible oil comprising long chain fatty acids and/or medium chain fatty acids and enhances the bioavailability of the nicotine compound; and
(c) an edible substrate. In some embodiments, the edible product is obtainable by the steps of:
(i) contacting the edible substrate with an oil comprising the nicotine compound and the bioavailability enhancing agent; and
(ii) dehydrating the edible substrate;
   thereby producing the edible product infused with a nicotine compound.

In some embodiments, the edible product is selected from the group consisting of a pill, tablet, lozenge, mini lozenge, capsule, caplet, pouch, gum, spray, food, and combinations thereof.

In one aspect, a food product infused with a nicotine compound is provided, comprising: (a) a therapeutically effective amount of a nicotine compound; (b) a bioavailability enhancing agent, wherein the bioavailability enhancing agent enhances the bioavailability of the nicotine compound; and (c) a food product, wherein the food product is selected from the group consisting of tea leaves, coffee beans, cocoa powder, meats, fish, fruits, vegetables, dairy products, legumes, pastas, breads, grains, seeds, nuts, spices, and herbs. In another aspect, the food product infused with a nicotine compound is obtainable by the steps of: (i) contacting the food product with an oil comprising the nicotine compound and the bioavailability enhancing agent; and (ii) dehydrating the food product; thereby producing the food product infused with the nicotine compound. In a further aspect, step (i) comprises saturating the food product in the oil comprising the nicotine compound and the bioavailability enhancing agent.

In another aspect, a beverage product infused with a nicotine compound is provided that is obtainable by the steps of: (i) providing tea leaves, coffee beans, or cocoa powder infused with the nicotine compound as described herein; and (ii) steeping the tea leaves, coffee beans, or cocoa powder infused with the nicotine compound in a liquid; thereby producing the beverage product infused with the nicotine compound.

In some embodiments, the edible product is a food product, wherein the edible substrate is selected from the group consisting of tea leaves, coffee beans, cocoa powder, meats, fish, fruits, vegetables, dairy products, legumes, pastas, breads, grains, seeds, nuts, spices, and herbs.

A. Nicotine Compounds

Nicotine is a natural ingredient in tobacco leaves where it acts as a botanical insecticide (Hukkanen et al. (2005) Pharmacological Reviews 57:79-115). Comprising about 95% of the total alkaloid content of commercial cigarette tobacco, nicotine comprises about 1.5% by weight of commercial cigarette tobacco (Hukkanen et al. (2005) Pharmacological Reviews 57:79-115). Although oral snuff and pipe tobacco contain concentrations of nicotine similar to cigarette tobacco, cigar and chewing tobacco typically contain only about half of the nicotine concentration of cigarette tobacco (Hukkanen et al. (2005) Pharmacological Reviews 57:79-115). An average tobacco rod typically contains 10 to 14 mg of nicotine (Hukkanen et al. (2005) Pharmacological Reviews 57:79-115), and on average about 1 to 1.5 mg of nicotine is absorbed systemically during smoking (Hukkanen et al. (2005) Pharmacological Reviews 57:79-115). The nicotine in tobacco is largely the levorotary (S)-isomer, only 0.1 to 0.6% of total nicotine content is (R)-nicotine (Hukkanen et al. (2005) Pharmacological Reviews 57:79-115). The (R)-nicotine content of tobacco smoke is higher, with up to 10% of nicotine in smoke reported to be (R)-isomer, and thought to be attributed to racemization occurring during combustion (Hukkanen et al. (2005) Pharmacological Reviews 57:79-115).

More than 99% of all nicotine that is consumed worldwide is delivered through smoking cigarettes. Approximately 6,000,000 deaths per year, worldwide, are attributed primarily to the delivery of nicotine through the act of smoking according to the Centers for Disease Control and Prevention, which also estimates that over $170 billion per year is spent just in the U.S. on direct medical care costs for adult smokers. In any twelve month period, 69% of U.S. adult smokers want to quit smoking and 43% of U.S. adult smokers have attempted to quit.

Worldwide, retail cigarette sales were worth $722 billion in 2013, with over 5.7 trillion cigarettes sold to more than 1 billion smokers. It would be desirable in the art to provide further methods for altering the character and nature of tobacco (and tobacco compositions and formulations) useful in smoking articles and/or or smokeless tobacco products, including enhancement of bioavailability of active agents, masking of unpleasant tastes, and the incorporation of additional active agents. Furthermore, the delivery of nicotine to satisfy current demand via the compositions and methods of the present invention, can in part alleviate the consumer demand for cigarettes. Since most of the adverse health outcomes of nicotine consumption are associated with the delivery method and only to a lesser degree to the actual ingestion of nicotine, a vast positive community health outcome can be achieved through the reduction in smoking cigarettes.

Accordingly, in other aspects, within the compositions and methods of the present invention, the lipophilic active agent is a nicotine compound.

As used herein, “nicotine compound” or “source of nicotine” often refers to naturally-occurring or synthetic nicotine compound unbound from a plant material, meaning the compound is at least partially purified and not contained within a plant structure, such as a tobacco leaf. Most preferably, nicotine is naturally-occurring and obtained as an extract from a Nicotiana species (e.g., tobacco). The nicotine can have the enantiomeric form S(−)-nicotine, R(+)-nicotine, or a mixture of S(−)-nicotine and R(+)-nicotine. Most preferably, the nicotine is in the form of S(−)-nicotine (e.g., in a form that is virtually all S(−)-nicotine) or a racemic mixture composed primarily or predominantly of S(−)-nicotine (e.g., a mixture composed of about 95 weight parts S(−)-nicotine and about 5 weight parts R(+)-nicotine). Most preferably, the nicotine is employed in virtually pure form or in an essentially pure form. Highly preferred nicotine that is employed has a purity of greater than about 95 percent, more preferably greater than about 98 percent, and most preferably greater than about 99 percent, on a weight basis. Despite the fact that nicotine can be extracted from Nicotiana species, it is highly preferred that the nicotine (and the composition and products produced in accordance with the present invention) are virtually or essentially absent of other components obtained from or derived from tobacco.

Nicotine compounds can include nicotine in free base form, salt form, as a complex, or as a solvate. See, for example, the discussion of nicotine in free base form in US Pat. Pub. No. 2004/0191322 to Hansson, which is incorporated herein by reference. At least a portion of the nicotine compound can be employed in the form of a resin complex of nicotine, where nicotine is bound in an ion exchange resin, such as nicotine polacrilex. See, for example, U.S. Pat. No. 3,901,248 to Lichtneckert et al., which is incorporated herein by reference. At least a portion of the nicotine can be employed in the form of a salt. Salts of nicotine can be provided using the types of ingredients and techniques set forth in U.S. Pat. No. 2,033,909 to Cox et al. and U.S. Pat. No. 4,830,028 to Lawson et al., and Perfetti, Beitrage Tabakforschung Int., 12: 43-54 (1983), which are incorporated herein by reference. See, also, U.S. patent application Ser. No. 12/769,335 to Brinkley et al., filed Apr. 28, 2010, which is incorporated herein by reference. Additionally, salts of nicotine have been available from sources such as Pfaltz and Bauer, Inc. and K&K Laboratories, Division of ICN Biochemicals, Inc.

Exemplary pharmaceutically acceptable nicotine salts include nicotine salts of tartrate (e.g., nicotine tartrate and nicotine bitartrate) chloride (e.g., nicotine hydrochloride and nicotine dihydrochloride), sulfate, perchlorate, ascorbate, fumarate, citrate, malate, lactate, aspartate, salicylate, tosylate, succinate, pyruvate, and the like; nicotine salt hydrates (e.g., nicotine zinc chloride monohydrate), and the like. Additional organic acids that can form salts with nicotine include formic, acetic, propionic, isobutyric, butyric, alpha-methylbutyric, isovaleric, beta-methylvaleric, caproic, 2-furoic, phenylacetic, heptanoic, octanoic, nonanoic, oxalic, malonic, and glycolic acid, as well as other fatty acids having carbon chains of up to about 20 carbon atoms.

In many embodiments, the nicotine compound will be present in multiple forms. For example, the nicotine can be employed within the composition as a mixture of at least two salts (e.g., two different organic acid salts, such as a mixture of nicotine bitartrate and nicotine levulinate), as at least two salts that are segregated within the composition, in a free base form and salt form, in a free base form and a salt form that are segregated within the composition, in a salt form and in a complexed form (e.g., a resin complex such as nicotine polacrilex), in a salt for and in a complexed form that are segregated with in the composition, in a free base form and a complexed form, in a free base form and a complexed form that are segregated within the composition, or the like. As such, each single dosage unit or piece (e.g., gum piece, lozenge, sachet, film strip, etc.) can incorporate at least two forms of nicotine.

A nicotine compound, in particular a compound such as nicotine, also can be employed in combination with other so-called tobacco alkaloids (i.e., alkaloids that have been identified as naturally occurring in tobacco). For example, nicotine, as employed in accordance with the present invention, can be employed in combination with nornicotine, anatabine, anabasine, and the like, and combinations thereof. See, for example, Jacob et al., Am. J. Pub. Health, 5: 731-736 (1999), which is incorporated herein by reference.

The compositions of the invention most preferably possess a form that is pharmaceutically effective and pharmaceutically acceptable. That is, the composition most preferably does not incorporate to any appreciable degree, or does not purposefully incorporate, significant amounts of components of tobacco, other than nicotine. As such, pharmaceutically effective and pharmaceutically acceptable compositions do not include tobacco in parts or pieces, processed tobacco components, or many of the components of tobacco traditionally present within tobacco-containing cigarettes, cigars, pipes, or smokeless forms of tobacco products. Highly preferred compositions that are derived by extracting naturally-occurring nicotine from tobacco include less than 5 weight percent of tobacco components other than nicotine, more often less than about 0.5 weight percent, frequently less than about 0.25 weight percent, and typically are entirely absent or devoid of components of tobacco, processed tobacco components, or components derived from tobacco, other than nicotine, based on the total weight of the composition.

In some embodiments, the nicotine compound is selected from the group consisting of nicotine and a nicotine derivative, wherein the nicotine derivative comprises a nicotine salt, a nicotine complex, a nicotine polacrilex, or combinations thereof.

Tobacco alkaloids include nicotine and nicotine-like or related pharmacologically active compounds such as nor-nicotine, lobeline and the like, as well as the free base substance nicotine and all pharmacologically acceptable salts of nicotine, including acid addition salts. “Nicotine compounds” as that term is used herein therefore includes all the foregoing tobacco alkaloids, as well as nicotine salts including but not limited to nicotine hydrogen tartrate and nicotine bitartrate dihydrate, as well as nicotine hydrochloride, nicotine dihydrochloride, nicotine sulfate, nicotine citrate, nicotine zinc chloride monohydrate, nicotine salicylate, nicotine oil, nicotine complexed with cyclodextrin, polymer resins such as nicotine polacrilex, nicotine resinate, and other nicotine-ion exchange resins, either alone or in combination.

The nicotine compounds also include nicotine analogs that include, but are not limited to the structures shown below for (s)-Nicotine, Nornicotine, (S)-Cotinine, B-Nicotyrine, (S)-Nicotene-N′-Oxide, Anabasine, Anatabine, Myosmine, B-Nornicotyrine, 4-(Methylamino)-1-(3-pyridyl)-1-butene (Metanicotine) cis or trans, N′-Methylanabasine, N′Methylanatabine, N′Methylmyosmine, 4-(Methylamino)-1-(3-pyridyl)-1-butanone (Pseudoxynicotine), and 2,3′-Bipyridyl (Hukkanen et al. (2005) Pharmacological Reviews 57:79-115):

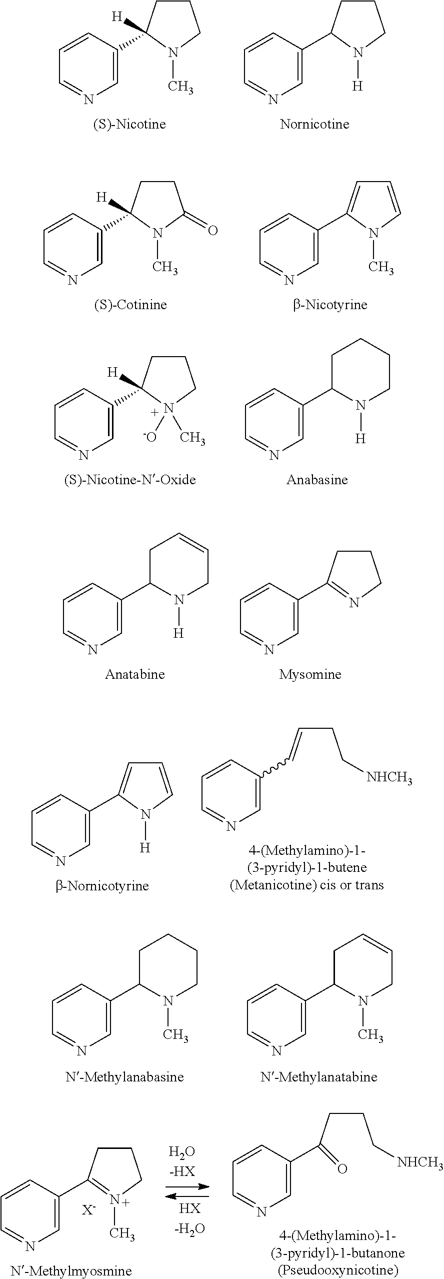

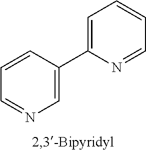 [SMILES: c1ccc(-c2cccnc2)nc1]

Nicotine compounds also include nicotine bitartrate, cytisine, nicotine polacrilex, nornicotine, nicotine 1-N-oxide, metanicotine, nicotine imine, nicotine N-glucuronide, N-methylnicotinium, N-n-decylnicotinium, 5′-cyanonicotine, 3,4-dihydrometanicotine, N′-methylnicotinium, N-octanoylnornicotine, 2,3,3a,4,5,9b-hexahydro-1-methyl-1H-pyrrolo(3,2-h)isoquinoline, 5-isothiocyanonicotine, 5-iodonicotine, 5′-hydroxycotinine-N-oxide, homoazanicotine, nicotine monomethiodide, N-4-azido-2-nitrophenylnornicotine, N-methylnornicotinium, nicotinium molybdophosphate resin, N-methyl-N′-oxonicotinium, N′-propylnornicotine, pseudooxynicotine, 4′-methylnicotine, 5-fluoronicotine, K(s-nic)5(Ga2(N,N′-bis-(2,3-dihydroxybenzoyl)-1,4-phenylenediamine)3), 5-methoxynicotine, 1-benzyl-4-phenylnicotinamidinium, 6-n-propylnicotine, SIB1663, 6-hydroxynicotine, N-methyl-nicotine, 6-(2-phenylethyl)nicotine, N′-formylnornicotine, N-n-octylnicotinium, N-(n-oct-3-enyl)nicotinium, N-(n-dec-9-enyl)nicotinium, 5′-acetoxy-N′-nitrosonornicotine, 4-hydroxynicotine, 4-(dimethylphenyl silyl)nicotine, N′-carbomethoxynornicotine, and N-methylnicoton.

The nicotine compound may be used in one or more distinct physical forms well known in the art, including free base forms, encapsulated forms, ionized forms and spray-dried forms.

Additional description regarding the chemistry, absorption, metabolism, kinetics and biomarkers of nicotine is described in Hukkanen et al. (2005) Pharmacological Reviews 57:79-115 and Benowitz et al. (2009) Handb. Exp. Pharmacol. 192:29-60, which are both incorporated herein in their entireties.

The compositions also include nicotine compounds characterized as selective agonists to nicotinic receptor subtypes that are present in the brain, or that can otherwise be characterized as a compound that modulates nicotinic receptor subtypes of the CNS. Various nicotinic receptor subtypes are described in Dwoskin et al., Exp. Opin. Ther. Patents, 10: 1561-1581 (2000); Huang et al., J. Am. Chem. Soc., 127: 14401-14414 (2006); and Millar, Biochem. Pharmacol., 78: 766-776 (2009); which are incorporated herein by reference. Representative compounds that can be characterized as other nicotine compounds for purposes of this invention are set forth in Schmitt et al., Annual Reports in Med. Chem. 35: 41-51 (2000); and Arneric et al., Biochem. Pharmacol., 74: 1092-1101 (2007); which are incorporated herein by reference.

In one aspect, the nicotine compound can be a compound has selectivity to the α₇ (alpha 7) nicotinic receptor subtype, and preferably is an agonist of the α₇ nicotinic receptor subtype. Several compounds having such α₇ receptor subtype selectivity have been reported in the literature. For example, various compounds purported to have selectivity to the α₇ nicotinic receptor subtype are set forth in Malysz et al., Assay Drug Dev. Tech., August: 374-390 (2009). An example of one such nicotine compound is N-[(2S,3S)-2-(pyridin-3-ylmethyl)-1-azabicyclo[2.2.2]oct-3-yl]-1-benzofuran-2-carboxamide (also known as TC-5619). See, for example, Hauser et al., Biochem. Pharmacol., 78: 803-812 (2009). Another representative is compound is (5aS,8S,10aR)-5a,6,9,10-Tetrahydro,7H,11H-8,10a-methanopyrido [2′,3′:5,6]pyrano[2,3-d]azepine (also known as dianicline or SSR591813 or SSR-591,813). See, for example, Hajos et al., J. Pharmacol. Exp. Ther., 312: 1213-1222 (2005). Another representative compound is 1,4-Diazabicyclo[3.2.2]nonane-4-carboxylic acid, 4-bromophenyl ester (also known as SSR180711). See, for example, Biton et al., Neuropsychopharmacol., 32: 1-16 (2007). Another representative compound is 3-[(3E)-3-[(2,4-dimethoxyphenyl)methylidene]-5,6-dihydro-4H-pyridin-2-yl]pyridine (also known as GTS-21). See, for example, U.S. Pat. No. 5,516,802 to Zoltewicz et al. and U.S. Pat. No. 5,741,802 to Kem et al. Another representative compound is 2-methyl-5-(6-phenyl-pyridazin-3-yl)-octahydro-pyrrolo[3,4-c]pyrrole (also known as A-582941). See, for example, Thomsen et al., Neuroscience, 154: 741-753 (2008). Another representative compound is (5S)-spiro[1,3-oxazolidine-5,8′-1-azabicyclo[2.2.2]octane]-2-one (also known as AR-R-17779 or AR-R-17779). See, for example, Li et al., Neuropsycopharmacol., 33: 2820-2830 (2008). Another representative compound is N-[(3R)-1-azabicyclo[2.2.2]oct-3-yl]-4-chlorobenzamide (also known as PNU-282,987). See, for example, Siok et al., Eur. J. Neurosci., 23: 570-574 (2006). Another representative compound is 5-morpholin-4-yl-pentanoic acid (4-pyridin-3-yl-phenyl)-amide (also known as WAY-317,538 or SEN-12333). See, for example, Roncarati et al., J Pharmacol. Exp. Ther., 329: 459-468 (2009). Yet other examples are compounds are those designated as EVP-6124 and EVP-4473 by Envivo Pharmaceuticals, Inc., TC-6987 by Targacept, Inc. and MEM3454 by Memory Pharmaceuticals Corp. The foregoing cited references are incorporated herein by reference.

In one aspect, the nicotine compound can be a compound that has selectivity to the α₄β₂ (alpha 4 beta 2) nicotinic receptor subtype, and preferably is an agonist of the α₄β₂ nicotinic receptor subtype. Several compounds having such α₄β₂ receptor subtype selectivity have been reported in the literature. An example of one such nicotine compound is known as 7,8,9,10-tetrahydro-6,10-methano-6H-pyrazino(2,3-h)(3) benzazepine (also known as varenicline and in the form of varenicline tartrate which is the active ingredient of a product commercially marketed under the tradename Chantix or Champix by Pfizer). See, for example, Jorenby et al., JAMA, 296: 56-63 (2006) and US Pat. Pub. No. 2010/0004451 to Ahmed et al. Another representative compound is (25,4E)-5-(5-isopropoxypyridin-3-yl)-N-methylpent-4-en-2-amine (also known as ispronicline or AZD-3480 of AstraZeneca or TC-1734 of Targacept, Inc. (Winston-Salem, N.C., USA)). See, for example, Dunbar et al., Psychopharmacol. (Berlin), 191: 919-929 (2007). Another representative compound is [3-(2(S))-azetidinylmethoxy)pyridine] dihydrochloride, (also known as A-85380). See, for example, Schreiber, Psychopharmacol., 159:248-257 (2002). Another representative compound is (5aS,8S,10aR)-5a,6,9,10-Tetrahydro,7H,11H-8,10a-methanopyrido [2′,3′:5,6]pyrano[2,3-d]azepine (also known as SSR591813). See, for example, Cohen et al., Neuroscience, Pres. No. 811.5 (2002); and Cohen et al., J. Pharmacol. Exp. Ther., 306: 407-420 (2003). Another representative compound is known as A-969933. See, for example, Zhu et al., Biochem. Pharmacol., 78: 920 (2009). Other representative compounds are known as S35836-1 and S35678-1. See, for example, Lockhart et al., Neuroscience, Pres. No. 684.9 (2002). Yet other examples are compounds are those designated as 3-(5,6-Dichloro-pyridin-3-yl)-1S,5S-3,6-diazabicyclo[3.2.0]heptane (also known as Sofinicline or ABT-894) by Abbott Laboratories; AZD1446 by AstraZeneca and TC-6499 by Targacept, Inc. The foregoing cited references are incorporated herein by reference.

In some cases, the nicotine can be liquid nicotine. Liquid nicotine can be purchased from commercial sources, whether tobacco-derived or synthetic. Tobacco-derived nicotine can include one or more other tobacco organoleptic components other than nicotine. The tobacco-derived nicotine can be extracted from raw (e.g., green leaf) tobacco and/or processed tobacco. Processed tobaccos can include fermented and unfermented tobaccos, dark air-cured, dark fire cured, burley, flue cured, and cigar filler or wrapper, as well as the products from the whole leaf stemming operation. The tobacco can also be conditioned by heating, sweating and/or pasteurizing steps as described in U.S. Publication Nos. 2004/0118422 or 2005/0178398. Fermenting typically is characterized by high initial moisture content, heat generation, and a 10 to 20% loss of dry weight. See, e.g., U.S. Pat. Nos. 4,528,993; 4,660,577; 4,848,373; and 5,372,149. By processing the tobacco prior to extracting nicotine and other organoleptic components, the tobacco-derived nicotine may include ingredients that provide a favorable experience. The tobacco-derived nicotine can be obtained by mixing cured tobacco or cured and fermented tobacco with water or another solvent (e.g., ethanol) followed by removing the insoluble tobacco material. The tobacco extract may be further concentrated or purified. In some cases, select tobacco constituents can be removed. Nicotine can also be extracted from tobacco in the methods described in the following patents: U.S. Pat. Nos. 2,162,738; 3,139,436; 3,396,735; 4,153,063; 4,448,208; and 5,487,792.

Liquid nicotine can be pure, substantially pure, or diluted prior to mixing it with soluble fiber. Soluble fiber dissolves in water at ambient temperature. Insoluble fiber does not dissolve in water at ambient temperature. Soluble fibers can attract water and form a gel. Not only are many soluble fibers safe for consumption, but some soluble fibers are used as a dietary supplement. As a dietary supplement, soluble fiber can slow down digestion and delay the emptying of a stomach. Instead of using soluble fiber as a mere additive, however, nicotine lozenges provided herein include a matrix of soluble fiber, which can dissolve to provide access to nicotine (and optionally other additives) included in the soluble-fiber matrix.

For liquid nicotine, a diluting step is optional. In some cases, liquid nicotine is diluted to a concentration of between 1 weight percent and 75 weight percent prior to mixing the liquid nicotine with soluble fiber. In some cases, liquid nicotine is diluted to a concentration of between 2 weight percent and 50 weight percent prior to mixing the liquid nicotine with soluble fiber. In some cases, liquid nicotine is diluted to a concentration of between 5 weight percent and 25 weight percent prior to mixing the liquid nicotine with soluble fiber. For example, liquid nicotine can be diluted to a concentration of about 10 weight percent prior to mixing the liquid nicotine with soluble fiber.

B. Edible Substrates

The term “edible substrate” means any edible material, hard or soft, including varying degrees of hardness or softness. Examples of suitable substrates include, but are not limited to, inulin, starch, modified starches, xanthan gum, carboxymethyl cellulose, methyl cellulose, hydroxypropylmethyl cellulose, konjac, chitosan, tragacanth, karaya, ghatti, larch, carageenan, alginate, chemically modified alginate, agar, guar, locust bean, psyllium, tara, gellan, curdlan, pullan, gum arabic, gelatin, pectin, and combinations thereof.

Other suitable edible substrates include chewing gum, bubble gum, fat based gum, such as described in U.S. Patent Application Publication No. US 20080057155, incorporated herein by reference, candy gum, including crunch gum and marshmallow gum such as described in U.S. Patent Application Publication Nos. US 20080166449 and US 20080199564, each incorporated herein by reference, relatively soft/hard gums which turn hard/soft or remain soft/hard after chewing, candy, chocolate and combinations thereof including gum and candy combinations including soft and hard layers or regions with varying degrees of crunchiness, a layer or region of layering material as defined above, any other edible material that can be employed in an edible composition, including hard or soft layers or regions of conventional materials applied by conventional methods, such as hard panning and soft panning, or the like.

Additional edible substrates include gum base, sticky gum substrates, as well as hygroscopic, moisture sensitive and/or heat sensitive substrates.

C. Flavoring Agents and Additives

In some embodiments, the edible product further comprises a flavoring agent selected from the group consisting of vanilla, vanillin, ethyl vanillin, orange oil, fruit and berry type flavorants, Dramboui, bourbon, scotch, whiskey, spearmint, lavender, cinnamon, chai, cardamon, apium graveolents, clove, cascarilla, nutmeg, sandalwood, bergamot, geranium, honey essence, rose oil, lemon oil, Japanese mint, cassia, caraway, cognac, jasmin, chamomile, menthol, ylang ylang, sage, fennel, pimenta, ginger, anise, chai, coriander, coffee, peppermint, wintergreen, mint oils from a species of the genus Mentha, and combinations thereof.

In some embodiments, the edible product further comprises an additive selected from the group consisting of a non-nicotine alkaloid, a mineral, a vitamin, a dietary supplement, a dietary mineral, a nutraceutical, an energizing agent, a soothing agent, a coloring agent, an amino acid, a chemsthetic agent, an antioxidant, a food grade emulsifier, a pH modifier, a botanical, a teeth whitening agent, a therapeutic agent, a sweetener, a flavorant, and combinations thereof.

D. Bioavailability

In some embodiments, the bioavailability of the nicotine compound in a subject is at least 2 times, 5 times, or 10 times greater than the bioavailability of the nicotine compound in the subject in the absence of the edible oil comprising long chain fatty acids and/or medium chain fatty acids. In some embodiments, the edible oil comprising long chain fatty acids and/or medium chain fatty acids is substantially free of omega-6 fatty acids. In some embodiments, the long chain fatty acids and/or medium chain fatty acids are selected from the group consisting of oleic acid, undecanoic acid, valeric acid, heptanoic acid, pelargonic acid, capric acid, lauric acid, and eicosapentaenoic acid.

In some embodiments, the bioavailability enhancing agent is a protective colloid, an edible oil or fat, and a lipophilic active agent taste masking agent. In some embodiments, the bioavailability enhancing agent that is a protective colloid, an edible oil or fat, and a lipophilic active agent taste masking agent is nonfat dry milk.

Bioavailability refers to the extent and rate at which the active moiety (drug or metabolite) enters systemic circulation, thereby accessing the site of action. Bioavailability for a given formulation provides an estimate of the relative fraction of the orally administered dose that is absorbed into the systemic circulation. Low bioavailability is most common with oral dosage forms of poorly water-soluble, slowly absorbed drugs. Insufficient time for absorption in the gastrointestinal tract is a common cause of low bioavailability. If the drug does not dissolve readily or cannot penetrate the epithelial membrane (e.g., if it is highly ionized and polar), time at the absorption site may be insufficient. Orally administered drugs must pass through the intestinal wall and then the portal circulation to the liver, both of which are common sites of first-pass metabolism (metabolism that occurs before a drug reaches systemic circulation). Thus, many drugs may be metabolized before adequate plasma concentrations are reached.

Bioavailability is usually assessed by determining the area under the plasma concentration-time curve (AUC). AUC is directly proportional to the total amount of unchanged drug that reaches systemic circulation. Plasma drug concentration increases with extent of absorption; the maximum (peak) plasma concentration is reached when drug elimination rate equals absorption rate. Peak time is the most widely used general index of absorption rate; the slower the absorption, the later the peak time.

The bioavailability of some drugs is increased when co-administered with food, particularly agents such as cannabinoids that are Class II drugs under the Biopharmaceutical Drug Classification System (Kelepu et al. (2013) Acta Pharmaceutica Sinica B 3:361-372; Amidon et al. (1995) Pharm. Res. 12:413-420; Charman et al. (1997) J. Pharm. Sci. 86:269-282; Winstanley et al. (1989) Br. J. Clin. Pharmacol. 28:621-628). It is the lipid component of the food that plays a key role in the absorption of lipophilic drugs and that leads to enhanced oral bioavailability (Hunt & Knox (1968) 1 Physiol. 194:327-336; Kelepu et al. (2013) Acta Pharmaceutica Sinica B 3:361-372). This has been attributed to the ability of a high fat meal to stimulate biliary and pancreatic secretions, to decrease metabolism and efflux activity, to increase intestinal wall permeability, and to a prolongation of gastrointestinal tract (GIT) residence time and transport via the lymphatic system (Wagnera et al. (2001) Adv. Drug Del. Rev. 50:S13-31; Kelepu et al. (2013) Acta Pharmaceutica Sinica B 3:361-372). High fat meals also elevate triglyceride-rich lipoproteins that associate with drug molecules and enhance intestinal lymphatic transport, which leads to changes in drug disposition and changes the kinetics of the pharmacological actions of poorly soluble drugs (Gershkovich et al. (2007) Eur. J. Pharm. Sci. 32:24-32; Kelepu et al. (2013) Acta Pharmaceutica Sinica B 3:361-372). However, co-administration of food with lipophilic drugs requires close control and/or monitoring of food intake when dosing such drugs, and can also be subject to problems with patient compliance (Kelepu et al. (2013) Acta Pharmaceutica Sinica B 3:361-372).

In other aspects, the bioavailability enhancing agent within the compositions and methods of the present invention is an edible oil or fat, a protective colloid, or both a protective colloid and an edible oil or fat. In another aspect, the bioavailability enhancing agent is also a lipophilic active agent taste masking agent. In another particular aspect, where the bioavailability enhancing agent is both a protective colloid, an edible oil or fat, and a lipophilic active agent taste masking agent, the bioavailability enhancing agent is nonfat dry milk. In a further aspect, the bioavailability enhancing agent is substantially free of omega-6 fatty acids. In other aspects, the bioavailability of the lipophilic active agent in a subject is at least about 1.5 times, 2 times, 5 times, or 10 times greater than the bioavailability of the lipophilic active agent in the subject in the absence of the bioavailability enhancing agent. In a further aspect, the bioavailability of the lipophilic active agent in a subject is greater than 20%.

An edible oil is defined herein as an oil that is capable of undergoing de-esterification or hydrolysis in the presence of pancreatic lipase in vivo under normal physiological conditions. Specifically, digestible oils may be complete glycerol triesters of medium chain (C₇-C₁₃) or long chain (C₁₄-C₂₂) fatty acids with low molecular weight (up to C₆) mono-, di- or polyhydric alcohols. Some examples of digestible oils for use in this invention thus include: vegetable, nut, or seed oils (such as coconut oil, peanut oil, soybean oil, safflower seed oil, corn oil, olive oil, castor oil, cottonseed oil, arachis oil, sunflower seed oil, coconut oil, palm oil, rapeseed oil, evening primrose oil, grape seed oil, wheat germ oil, sesame oil, avocado oil, almond, borage, peppermint and apricot kernel oils) and animal oils (such as fish liver oil, shark oil and mink oil).

In a further aspect, the bioavailability enhancing agent is a long chain (C₁₄-C₂₂) fatty acid. In a further aspect, the bioavailability enhancing agent is a medium chain (C₇-C₁₃) fatty acid. In further aspects, the bioavailability enhancing agent is a combination of medium and long chain fatty acids.

Examples of protective colloids include polypeptides (such as gelatin, casein, and caseinate), polysaccharides (such as starch, dextrin, dextran, pectin, and gum arabic), as well as whole milk, skimmed milk, milk powder or mixtures of these. However, it is also possible to use polyvinyl alcohol, vinyl polymers, for example polyvinylpyrrolidone, (meth)acrylic acid polymers and copolymers, methylcellulose, carboxymethylcellulose, hydroxypropylcellulose and alginates. For further details, reference may be made to R. A. Morton, Fast Soluble Vitamins, Intern. Encyclopedia of Food and Nutrition, Vol. 9, Pergamon Press 1970, pages 128-131.

Oral administration constitutes the preferred route of administration for a majority of drugs. However, drugs that have an undesirable or bitter taste leads to lack of patient compliance in the case of orally administered dosage forms. In such cases, taste masking is an essential tool to improve patient compliance. Because lipophilic active agents (e.g., nicotine compounds) have an undesirable taste profile, in order to improve compliance, the presently disclosed compositions also comprise one or more lipophilic active agent taste masking agents. Examples of lipophilic active agent taste-masking agents include dry milk as described above, as well as menthol, sweeteners, sodium bicarbonate, ion-exchange resins, cyclodextrin inclusion compounds, adsorbates, and the like.

In another aspect, taste-masking agents used with the food and beverage products infused with a nicotine compound of the present invention may further include flavoring agents such as salts (e.g., sodium chloride, potassium chloride, sodium citrate, potassium citrate, sodium acetate, potassium acetate, and the like), natural sweeteners (e.g., fructose, sucrose, glucose, maltose, mannose, galactose, lactose, and the like), artificial sweeteners (e.g., sucralose, saccharin, aspartame, acesulfame K, neotame, and the like); and mixtures thereof. In other aspects, suitable flavoring agents include, but are not limited to, vanilla, vanillin, ethyl vanillin, orange oil, fruit and berry type flavorants, Dramboui, bourbon, scotch, whiskey, spearmint, lavender, cinnamon, chai, cardamon, apium graveolents, clove, cascarilla, nutmeg, sandalwood, bergamot, geranium, honey essence, rose oil, lemon oil, Japanese mint, cassia, caraway, cognac, jasmin, chamomile, menthol, ylang ylang, sage, fennel, pimenta, ginger, anise, chai, coriander, coffee, peppermint, wintergreen, mint oils from a species of the genus Mentha, and combinations thereof.

In a further aspect, the bioavailability enhancing agent is substantially free of omega-6 fatty acids. As used herein, “substantially free” means largely but not wholly pure. For example, “substantially free” means less than 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, or 1.0% by weight.

In other aspects, the bioavailability of the lipophilic active agent in a subject is at least about 1.5 times, 2 times, 2.5 times, 3 times, 3.5 times, 4 times, 4.5 times, 5 times, 5.5 times, 6 times, 6.5 times, 7 times, 7.5 times, 8 times, 8.5 times, 9 times, 9.5 times, or 10 times greater than the bioavailability of the lipophilic active agent in the subject in the absence of the bioavailability enhancing agent.

In a further aspect, the bioavailability of the lipophilic active agent in a subject is greater than 20% or at least about 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or greater.

Assays and methods for measuring lipophilic active agent bioavailability are well known in the art (see, e.g., Rocci & Jusko (1983) Compd. Programs Biomed. 16:203-215; Shargel & Yu (1999) Applied biopharmaceutics & pharmacokinetics (4th ed.). New York: McGraw-Hill; Hu & Li (2011) Oral Bioavailability: Basic Principles, Advanced Concepts, and Applications, John Wiley & Sons Ltd.; Karschner et al. (2011) Clinical Chemistry 57:66-75; Ohlsson et al. (1980) Clin. Pharmacol. Ther. 28:409-416; Ohlsson et al. (1982) Biomed. Environ. Mass Spectrom. 9:6-10; Ohlsson et al. (1986) Biomed. Environ. Mass Spectrom. 13:77-83; Karschner et al. (2010) Anal. Bioanal. Chem. 397:603-611).

E. Secondary Active Agents

In some embodiments, the edible product further comprises a secondary active agent, wherein the secondary active agent is a lipophilic active agent selected from the group consisting of: cannabinoids, terpenes and terpenoids, non-steroidal anti-inflammatory drugs (NSAIDs), vitamins, nicotine or an analog thereof, phosphodiesterase 5 (PDE5) inhibitors, Maca extract, hormones, fentanyl or an analog thereof, buprenorphine or an analog thereof, scopolamine or an analog thereof, and antioxidants. In some embodiments, the cannabinoid is a psychoactive cannabinoid. In some embodiments, the cannabinoid is a non-psychoactive cannabinoid. In some embodiments, the NSAID is acetylsalicylic acid, ibuprophen, acetaminophen, diclofenac, indomethacin, piroxicam, or a COX inhibitor. In some embodiments, the vitamin is vitamin A, D, E, or K. In some embodiments, the PDE5 inhibitor is avanafil, lodenafil, mirodenafil, sildenafil, tadalafil, vardenafil, udenafil, acetildenafil, thiome-thisosildenafil, or analogs thereof. In some embodiments, the hormone is an estrogen, an anti-estrogen, an androgen, an anti-androgen, or a progestin. In some embodiments, the antioxidant is astaxanthin, Superoxide Dismusase, beta-carotene, selenium, lycopene, lutein, Coenzyme Q10, phytic acid, flavonoids, a polyphenol, a substituted 1,2-dihydroquinoline, ascorbic acid and its salts, ascorbyl palmitate, ascorbyl stearate, anoxomer, N-acetylcysteine, benzyl isothiocyanate, o-, m- or p-amino benzoic acid (o is anthranilic acid, p is PABA), butylated hydroxyanisole (BHA), butylated hydroxytoluene (BHT), caffeic acid, canthaxantin, alpha-carotene, beta-carotene, beta-caraotene, beta-apo-carotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids, flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, ice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., lonox 100), 2,4-(tris-3′,5′-bi-tert-butyl-4′-hydroxybenzyl)-mesitylene (i.e., lonox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivates, vitamin Q10, wheat germ oil, zeaxanthin, or combinations thereof.

F. Lyophylization

In a further aspect, the food product infused with a nicotine compound of the present invention is lyophilized. Lyophilization, also known as freeze-drying, is a process whereby water is sublimed from a composition after it is frozen. The frozen solution is then typically subjected to a primary drying step in which the temperature is gradually raised under vacuum in a drying chamber to remove most of the water, and then to a secondary drying step typically at a higher temperature than employed in the primary drying step to remove the residual moisture in the lyophilized composition. The lyophilized composition is then appropriately sealed and stored for later use. Tang et al. (2004) Pharmaceutical Research 21:191-200 describes the scientific principles pertaining to freeze drying and guidelines for designing suitable freeze drying processes. Further description of freeze drying is found in Remington (2006) The Science and Practice of Pharmacy, 21^{st} edition, Lippincott Williams & Wilkins, pp. 828-831.

### II. Processes

In some embodiments, a process for making an edible product infused with a nicotine compound is provided, comprising:
(a) providing a therapeutically effective amount of a nicotine compound;
(b) providing a bioavailability enhancing agent, wherein the bioavailability enhancing agent comprises an edible oil comprising long chain fatty acids and/or medium chain fatty acids and enhances the bioavailability of the nicotine compound;
(c) providing an edible substrate;
(di) contacting the edible substrate with an oil comprising the nicotine compound and the bioavailability enhancing agent; and
(e) dehydrating the edible substrate;
   thereby producing the edible product infused with a nicotine compound.

In some embodiments, a beverage product infused with a nicotine compound is provided obtainable by the steps of:
(i) providing the edible product infused with a nicotine compound as described above, wherein the edible product infused with a nicotine compound is tea leaves, coffee beans, or cocoa powder infused with a nicotine compound; and
(ii) steeping the tea leaves, coffee beans, or cocoa powder infused with a nicotine compound in a liquid;
   thereby producing the beverage product infused with the nicotine compound. In some embodiments, the beverage product infused with a nicotine compound is obtainable by the steps of:
(i) providing the edible product infused with a nicotine compound as described above, wherein the edible product infused with a nicotine compound is tea leaves, coffee beans, or cocoa powder infused with a nicotine compound; and
(ii) steeping the tea leaves, coffee beans, or cocoa powder infused with a nicotine compound in a liquid;
   thereby producing the beverage product infused with the nicotine compound.

In other aspects, a process for making a food product infused with a nicotine compound is provided comprising the steps of: (i) contacting a food product with an oil comprising a nicotine compound and a bioavailability enhancing agent; and (ii) dehydrating the food product; thereby producing the food product infused with the nicotine compound; wherein the food product infused with the nicotine compound comprises a therapeutically effective amount of the nicotine compound, and further wherein: (a) the bioavailability enhancing agent enhances the bioavailability of the lipophilic active agent; and (b) the food product is selected from the group consisting of tea leaves, coffee beans, cocoa powder, meats, fish, fruits, vegetables, dairy products, legumes, pastas, breads, grains, seeds, nuts, spices, and herbs. In another aspect, step (i) comprises saturating the food product in the oil comprising the nicotine compound and the bioavailability enhancing agent. In another aspect, step (i) further comprises contacting the food product with a flavoring agent, particularly wherein the flavoring agent is selected from the group consisting of vanilla, vanillin, ethyl vanillin, orange oil, fruit and berry type flavorants, Dramboui, bourbon, scotch, whiskey, spearmint, lavender, cinnamon, chai, cardamon, apium graveolents, clove, cascarilla, nutmeg, sandalwood, bergamot, geranium, honey essence, rose oil, lemon oil, Japanese mint, cassia, caraway, cognac, jasmin, chamomile, menthol, ylang ylang, sage, fennel, pimenta, ginger, anise, chai, coriander, coffee, peppermint, wintergreen, mint oils from a species of the genus Mentha, and combinations thereof. In another aspect, the process further comprises a step of lyophilizing the food product infused with the nicotine compound.

In a further aspect, where the food product infused with the nicotine compound is tea leaves, coffee beans, or cocoa powder infused with the nicotine compound, the process further comprises packaging the tea leaves, coffee beans, or cocoa powder infused with the nicotine compound in single or multiple serve delivery devices, such as tea bags, water permeable membranes, pre-packaged beverage pods such as K-CUP® packs manufactured and sold by Keurig Inc. of Wakefield, Mass., and the like. Examples include, but are not limited to, such delivery devices and related systems as described in U.S. Pat. Nos. 3,450,024; 5,325,765; 5,840,189; and 6,606,938. In a particular aspect, the food product infused with the nicotine compound is tea leaves and the process further comprises packaging the tea leaves in tea bags.

In another aspect, a process for making a beverage product infused with a nicotine compound is provided comprising making tea leaves, coffee beans, or cocoa powder infused with the nicotine compound according to any of the processes described herein; further comprising the step of steeping the tea leaves, coffee beans, or cocoa powder infused with the nicotine compound in a liquid, thereby producing the beverage product infused with the nicotine compound. In further aspects, the disclosed processes and methods use dehydration methods using dielectric energy, particularly microwave energy. In some aspects, the dielectric energy is selected from the group consisting of radio frequency energy, low frequency microwave energy, and high frequency microwave energy. In some aspects, the dehydration methods further comprise using dielectric energy under vacuum. In still further aspects, the dehydration methods further comprise stirring at a temperature of less than 70° C. In still further aspects, the disclosed processes and methods use dehydration methods using spray drying technology (e.g., methods of producing dry powders from a liquid or slurry by rapidly drying with a hot gas; see generally Mujumdar (2007) Handbook of Industrial Drying, CRC Press).

### III. Methods of Treatment

In some embodiments, a method of treating a nicotine-related disorder is provided comprising administering the edible product infused with a nicotine compound or the beverage product infused with a nicotine compound to a subject in need thereof, and wherein the nicotine-related disorder is selected from the group consisting of tobacco dependence/addiction, Parkinson's disease, ulcerative colitis, Alzheimer's disease, schizophrenia, Attention Deficit Hyperactivity Disorder (ADHD), Tourette's syndrome, ulcerous colitis, and post-smoking-cessation weight control.

The nicotine compounds of the present invention are effective over a wide dosage range. For example, in treating adult humans, compositions and methods of the present invention comprise dosages of nicotine compounds from 0.01 mg to 1,000 mg, from 0.5 mg to 500 mg, from 1 mg to 100 mg, from 5 mg to 50 mg, and from 10 mg to 25 mg. Alternatively, in treating adult humans, compositions and methods of the present invention comprise dosages of nicotine compounds of 0.01 mg, 0.05 mg, 0.1 mg, 0.25 mg, 0.5 mg, 0.75 mg, 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1,000 mg.

In some embodiments, a method of enhancing the bioavailability of a nicotine compound is provided, comprising heating the edible product infused with a nicotine compound or the beverage product infused with a nicotine compound to a temperature that is greater than or equal to human body temperature. In some embodiments, the method of enhancing the bioavailability of a lipophilic active agent comprises oral administration of the edible product infused with a nicotine compound or the beverage product infused with a nicotine compound to a human subject.

In another aspect, a method of administering any of the nicotine compounds described herein to a subject is provided, comprising oral administration of any of the compositions of the present invention to the subject. Such administration may be for any purpose, including overall health and wellness, mental acuity, alertness, recreation, and the like.

As used herein, the term “subject” treated by the presently disclosed methods in their many aspects is desirably a human subject, although it is to be understood that the methods described herein are effective with respect to all vertebrate species, which are intended to be included in the term “subject.” Accordingly, a “subject” can include a human subject for medical purposes, such as for the diagnosis or treatment of an existing disease, disorder, condition or the prophylactic diagnosis or treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some aspects, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

The term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition, or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

Actual dosage levels of the active ingredients in the presently disclosed compositions can be varied so as to obtain an amount of the active ingredient that is effective to achieve the desired therapeutic response for a particular subject, composition, route of administration, and disease, disorder, or condition without being toxic to the subject. The selected dosage level will depend on a variety of factors including the activity of the particular composition employed, the route of administration, the time of administration, the rate of excretion of the particular composition being employed, the duration of the treatment, other drugs, and/or materials used in combination with the particular composition employed, the age, sex, weight, condition, general health and prior medical history of the patient being treated, and like factors well known in the medical arts.

A physician having ordinary skill in the art can readily determine and prescribe the effective amount of the presently disclosed composition required. Accordingly, the dosage range for administration may be adjusted by the physician as necessary, as described more fully elsewhere herein.

### IV. Kits and Containers

Also contemplated are kits that include any one of the compositions disclosed throughout the specification and claims. In certain embodiments, the composition is comprised in a container. The container can be a bottle, dispenser, or package. The container can dispense a pre-determined amount of the composition. The container can include indicia on its surface. The indicia can be a word, an abbreviation, a picture, or a symbol.

### EXAMPLES

### Example 1

A line of CBD and/or THC infused tea bags coming in a variety of flavors was developed.

I. Ingredients

Tea in leaf form, oil form, brewed form, organic and inorganic

Evaporated dry non-fat milk

CBD oil

Hemp oil or compatible oil for ingestion

Cannabis leaves, buds, oils; all strains with THC and/or CBD

II. ViPova® Formulas

II A. CBD Tea

Combine evaporated nonfat, dry milk with any and all teas, organic and inorganic

Blend CBD oil with the tea leaves

Dehydrate mixture of tea, CBD oil, and evaporated nonfat dry milk in a food dehydrator

End-product is ViPova® Tea with CBD enhancement only

II B. THC/CBD Tea

Combine evaporated nonfat, dry milk with any and all teas, organic and inorganic

Blend hemp or other ingestible oil with the tea leaves

Add cannabis leaves to above mixture

Dehydrate mixture of tea, hemp or other ingestible oil, cannabis leaves, and evaporated nonfat dry milk

End-product is ViPova® Tea with THC and CBD

III. ViPova® Formulas: Specifications

III A. CBD Tea

Tea: one tea bag contains 1 gram to 3 grams of tea leaves (dry weight)

Evaporated dry non-fat milk: 0.10-1.00 grams

CBD oil: 10 mgs.-25 mgs. per tea bag

III B. THC/CBD Tea

Tea: one tea bag contains 1.5-12 grams tea leaves (dry weight) per tea bag

Evaporated dry milk: 0.10-6.00 grams per tea bag

Hemp oil or other ingestible oil: 10 mgs.-25 mgs. per tea bag

Cannabis leaves: 1.00-12.00 grams per tea bag

III C. Production Equipment:

Commercial grinder for tea and/or cannabis leaves

Commercial mixer

Commercial dehydrator

Commercial tea bag filling machine

IV. Flavorings

ViPova® Teas will provide a menu of flavorings for addition to tea bags or loose tea selections including, but not limited to mint, citrus, and vanilla.

### Example 2

A process for adhering CBD and/or THC to food products was developed. The food products may be selected from the group consisting of meats, fish, fruits, vegetables, dairy products, legumes, pastas, breads, grains, seeds, nuts, spices, and herbs. The process may or may not involve contacting the food product with sunflower and/or dry evaporated milk. The process involved the steps of:

1. A food product was saturated with 0-60 grams of CBD and/or THC oil or extract.

2. The food product was placed on dehydrator paper and placed in a food dehydrator for 0-24 hours.

3. The food product was removed from the dehydrator and stored in air-tight containers.

### Example 3

Black tea was formulated with various lipophilic active agents. Active agents were dosed into the tea at a concentration of approximately 4.5 mg of active ingredient per gram of finished product, using non-fat dry milk and sunflower seed oil as excipients. The following ingredients were used for the formulation:

453 g of loose leaf black tea

2265 mg active agent

45 g of instant non-fat dry evaporated milk

1132.5 mg of sunflower seed oil

Ingredients were combined in a stainless steel bowl and mixed with gloved hands. A homogenous mixture was spread evenly on a dehydrator tray and dehydrated for 30 minutes. After cooling, the formulated tea was placed into a sterile zip-lock bag.

The active ingredients that were formulated were: ASA (aspirin), ibuprofen, acetaminophen, diclofenac, indomethacin, piroxicam, nicotine, and vitamin E (α-tocopherol). The specific supplier information and lot numbers for each active agent are shown below in Table 1.

TABLE 1
Active Agents Used for Formulations

| | | | Catalogue | |
|---|---|---|---|---|
| Compound | CAS Number | Supplier | Number | Lot Number |
| ASA | 50-78-2 | Sigma- | A2093 | #MKBQ8444V |
| (aspirin) | | Aldrich | | |
| Ibuprofen | 15687-27-1 | Sigma- | I4883 | #MKBQ4505V |
| | | Aldrich | | |
| Acetaminophen | 103-90-2 | Sigma- | A5000 | #MKBS7142V |
| | | Aldrich | | |
| Diclofenac | 15307-79-6 | Sigma- | D6899 | #BCBN3367V |
| | | Aldrich | | |
| Indomethacin | 53-86-1 | Sigma- | I8280 | #MKBR4530V |
| | | Aldrich | | |
| Piroxicam | 36322-90-4 | Sigma- | P0847 | #SLBF3478V |
| | | Aldrich | | |
| Nicotine | 54-11-5 | Sigma- | N3876 | #1449194V |
| | | Aldrich | | |
| Vitamin E (α- | 10191-41-0 | Sigma- | 258024 | #MKBT5983V |
| tocopherol) | | Aldrich | | |

The Tea used was loose leaf English Breakfast Tea from Upton Tea Imports (Holliston, Mass.).

The Sunflower Oil was Whole Foods brand organic sunflower oil.

The non-fat dry milk power was NowFoods brand organic non-fat dry milk.

The dehydrator used was a Presto Dehydrator, model #06300.

Each component of the formulation was weighed out and combined as described in the above procedure. The weights of the individual active agents for each formulation are summarized below in Table 2.

TABLE 2
Formulation of Active Agents

| | Compound | Non-Fat | Sunflower | | | Compound |
|---|---|---|---|---|---|---|
| Compound | Weight | Dry Milk | Seed Oil | Black Tea | Yield | Concentration |
| ASA (aspirin) | 2267.1 mg | 45.09 g | 1135 mg | 453.2 g | 479.3 g | 4.52 mg/g |
| Ibuprofen | 2265.5 mg | 45.05 g | 1138 mg | 453.8 g | 488.1 g | 4.51 mg/g |
| Acetaminophen | 2264.7 mg | 45.01 g | 1136 mg | 453.2 g | 477.9 g | 4.51 mg/g |
| Diclofenac | 2265.3 mg | 45.06 g | 1133 mg | 453.1 g | 441.3 g | 4.52 mg/g |
| Indomethacin | 2266.3 mg | 44.99 g | 1138 mg | 453.1 g | 491.5 g | 4.52 mg/g |
| Piroxicam | 2265.9 mg | 45.25 g | 1134 mg | 453.6 g | 488.3 g | 4.51 mg/g |
| Nicotine | 2264.9 mg | 45.02 g | 1133 mg | 453.1 g | 488.1 g | 4.52 mg/g |
| Vitamin E (α- | 2271.1 mg | 45.05 g | 1135 mg | 453.2 g | 480.2 g | 4.53 mg/g |
| tocopherol) | | | | | | |

For each formulation, the constituents were mixed by hand until a homogeneous mixture was achieved, then spread evenly on dehydrator trays for drying. Each formulation was dried for 30 minutes in dehydrator. After cooling, mixture was placed into Zip-Lock bag. After taring the analytical balance for the Zip-Lock bag, the weight of the final formulation was recorded and the concentration of active ingredient in the formulation calculated (Table 2).

### Example 4

As used herein, compositions incorporating DEHYDRATECH™ are compositions that incorporate a dehydrated mixture comprising a therapeutically effective amount of a lipophilic active agent and an edible oil comprising long chain fatty acids, particularly wherein dehydrated mixture is obtainable by the steps of:
i) combining a therapeutically effective amount of the lipophilic active agent with the edible oil comprising long chain fatty acids; and
ii) dehydrating the product of step (i), thereby producing the dehydrated mixture.

This study was designed to principally assess the relative ingestible nicotine absorption performance of DEHYDRATECH™-powered formulations compared to concentration-matched control formulations that lacked any form of delivery enabling technology in rats. Nicotine was administered in a nicotine polacrilex derivative format as is widely commercialized today in nicotine replacement therapy products such as chewing gums. Twelve male rats were divided into four groups of three, such that DEHYDRATECH™ and control formulations were each tested at a 1 mg/Kg and 10 mg/Kg dosage level. Formulations were administered orally and all rats were cannulated for blood collection at multiple intervals over an 8 hour duration post-dosing with the first data collection at the 15-minute mark. Urine and feces were also collected for up to a 24-hour duration post-dosing, and essential organ tissue samples were also collected for examination after the study. All samples were subjected to analytical testing in order to quantify the levels of nicotine therein, as well as the levels of three major liver metabolites thereof, hydroxycotinine, nicotine N′-oxide and cotinine, in order to assess the relative metabolite levels absorbed by the different formulations.

### Results & Observations

The DEHYDRATECH™ formulations generally achieved faster absorption, higher peak absorption and higher overall quantities of nicotine, on average, in the blood than the concentration-matched control formulations at both the 1 mg and 10 mg/Kg doses tested. Furthermore, as previously reported, there were no obvious signs of gastrointestinal distress such as vomiting or diarrhea indicating that the animals appeared to tolerate the treatment well.

Nicotine blood levels were evaluated multiple times over a period of 8 hours after dosing. In the 10 mg/Kg dosing arm, the control formulation required nearly 3 hours to reach similar levels of blood absorption that the DEHYDRATECH™ formulation reached in only 15 minutes. Furthermore, the DEHYDRATECH™ formulation went on thereafter to demonstrate peak plasma levels that were 148% of those achieved by the control formulation. If replicated in human studies, these findings are suggestive that DEHYDRATECH™'s technology could prove more effective in elevating blood nicotine levels through edible formats much more quickly and substantially than previously theorized, potentially making ingestible nicotine preparations a viable alternative to today's available product formats while also leading to a more rapid nicotine craving satiation.

Analysis of the liver metabolites revealed, as expected, that overall levels in the blood of two of the three metabolites studied were higher in the control group than in the DEHYDRATECH™ formulation group at the 10 mg/Kg dose. This result was especially pronounced in the 45-minute to 2-hour time interval post-dosing which is consistent with the expected timing of release of metabolites in higher quantity into the bloodstream by the liver following normal physiological processing of ingested nicotine with the control preparation, compared to the DehydraTECH™ technology that is believed to elude first pass liver metabolism. The DEHYDRATECH™ formulation also demonstrated lower quantities of nicotine in the rat urine at both doses, which is consistent with the fact that the levels of nicotine in the rat blood remained higher over the duration of the study with the DEHYDRATECH™ formulation than with the control. The study also revealed that the DEHYDRATECH™ formulation at the 10 mg/Kg level achieved up to 5.6-times as much nicotine upon analysis of the rat brain tissue than was recovered with the matching control formulation. These findings together perhaps suggest prolongation of nicotine effectiveness with the DEHYDRATECH™ formulation which may also be beneficial in humans to control cravings over an extended time-period from a single edible nicotine dose.

### Example 5

In this study, the exposure and distribution of nicotine and its major metabolites were evaluated following oral administration of two separate formulations (Reference and Test Nicotine Polacrilex) in male Sprague-Dawley rats.

Formulations were administered orally (PO) at 10 mg/kg. Following dosing, blood samples were collected up to 1 hour post dose; and urine and fecal samples were collected up to 24 hours post dose. Brain, liver, and kidney tissue were collected at 1 hour (Groups 1 & 5), 4 hours (Groups 2 & 6), following the 8 hour urine and feces sample collection (Groups 3 & 7), or following the 24 hour urine and feces sample collection (Groups 4 & 8). Blood, urine, feces, and tissue concentrations of each analyte were determined by LC-MS/MS. Plasma pharmacokinetic parameters were determined using WinNonlin (v8.0). Brain, liver, and kidney pharmacokinetic parameters were determined using WinNonlin (v8.0) software with sparse sampling.

Following PO dosing of Reference Nicotine Polacrilex at 10 mg/kg (Group 1), maximum plasma concentrations (average of 144±68.2 ng/mL) of nicotine were observed between 30 minutes and 1 hour post dosing. The average half-life after oral dosing could not be determined. The average exposure for nicotine (Group 1) based on the dose normalized AUClast was 8.71±2.76 hr*kg*ng/mL/mg.

Following PO dosing of Reference Nicotine Polacrilex at 10 mg/kg (Group 1), maximum plasma concentrations (average of 9.79±3.56 ng/mL) of hydroxycotinine metabolite were observed between 45 minutes and 1 hour post dosing. The average half-life after oral dosing could not be determined. The average exposure for hydroxycotinine (Group 1) based on the dose normalized AUClast was 0.420±0.146 hr*kg*ng/mL/mg.

Following PO dosing of Reference Nicotine Polacrilex at 10 mg/kg (Group 1), maximum plasma concentrations (average of 179±54.9 ng/mL) of nicotine-n-oxide metabolite were observed between 30 minutes and 1 hour post dosing. The average half-life after oral dosing could not be determined. The average exposure for nicotine-n-oxide (Group 1) based on the dose normalized AUClast was 11.2±3.32 hr*kg*ng/mL/mg.

Following PO dosing of Reference Nicotine Polacrilex at 10 mg/kg (Group 1), maximum plasma concentrations (average of 193±58.6 ng/mL) of cotinine metabolite were observed at 1 hour post dosing. The average half-life after oral dosing could not be determined. The average exposure for cotinine (Group 1) based on the dose normalized AUClast was 10.9±2.90 hr*kg*ng/mL/mg.

Following PO dosing of Reference Nicotine Polacrilex at 10 mg/kg (Group 2), maximum plasma concentrations (average of 350±256 ng/mL) of nicotine were observed between 8 minutes and 1 hour post dosing. The average half-life after oral dosing could not be determined. The average exposure for nicotine (Group 2) based on the dose normalized AUClast was 21.3±13.7 hr*kg*ng/mL/mg.

Following PO dosing of Reference Nicotine Polacrilex at 10 mg/kg (Group 2), maximum plasma concentrations (average of 20.1±13.3 ng/mL) of hydroxycotinine metabolite were observed at 1 hour post dosing. The average half-life after oral dosing could not be determined. The average exposure for hydroxycotinine (Group 2) based on the dose normalized AUClast was 1.15±0.928 hr*kg*ng/mL/mg.

Following PO dosing of Reference Nicotine Polacrilex at 10 mg/kg (Group 2), maximum plasma concentrations (average of 409±235 ng/mL) of nicotine-n-oxide metabolite were observed between 12 minutes and 1 hour post dosing. The average half-life after oral dosing could not be determined. The average exposure for nicotine-n-oxide (Group 2) based on the dose normalized AUClast was 26.8±18.3 hr*kg*ng/mL/mg.

Following PO dosing of Reference Nicotine Polacrilex at 10 mg/kg (Group 2), maximum plasma concentrations (average of 359±236 ng/mL) of cotinine metabolite were observed between 45 minutes and 1 hour post dosing. The average half-life after oral dosing could not be determined. The average exposure for cotinine (Group 2) based on the dose normalized AUClast was 22.5±16.7 hr*kg*ng/mL/mg.

Following PO dosing of Reference Nicotine Polacrilex at 10 mg/kg (Group 3), maximum plasma concentrations (average of 176±71.2 ng/mL) of nicotine were observed between 30 minutes and 1 hour post dosing. The average half-life after oral dosing could not be determined. The average exposure for nicotine (Group 3) based on the dose normalized AUClast was 11.7±4.62 hr*kg*ng/mL/mg. On average, 1.04±0.49% and 0.03±0.04% of the dose (unchanged dose) was found in urine and feces, respectively, after PO dosing.

Following PO dosing of Reference Nicotine Polacrilex at 10 mg/kg (Group 3), maximum plasma concentrations (average of 13.4±5.95 ng/mL) of hydroxycotinine metabolite were observed between 45 minutes 1 hour post dosing. The average half-life after oral dosing could not be determined. The average exposure for hydroxycotinine (Group 3) based on the dose normalized AUClast was 0.672±0.386 hr*kg*ng/mL/mg. On average, 1.10±0.64% and 0.03% (n=1) of the dose was found in urine and feces, respectively, after PO dosing.

Following PO dosing of Reference Nicotine Polacrilex at 10 mg/kg (Group 3), maximum plasma concentrations (average of 283±134 ng/mL) of nicotine-n-oxide metabolite were observed between 30 minutes and 1 hour post dosing. The average half-life after oral dosing could not be determined. The average exposure for nicotine-n-oxide (Group 3) based on the dose normalized AUClast was 17.8±7.29 hr*kg*ng/mL/mg. On average, 9.36±4.36% and 0.07% (n=1) of the dose was found in urine and feces, respectively, after PO dosing.

Following PO dosing of Reference Nicotine Polacrilex at 10 mg/kg (Group 3), maximum plasma concentrations (average of 304±103 ng/mL) of cotinine metabolite were observed at 1 hour post dosing. The average half-life after oral dosing could not be determined. The average exposure for cotinine (Group 3) based on the dose normalized AUClast was 15.4±4.99 hr*kg*ng/mL/mg. On average, 0.99±0.48% and 0.03±0.02% of the dose was found in urine and feces, respectively, after PO dosing.

Following PO dosing of Reference Nicotine Polacrilex at 10 mg/kg (Group 4), maximum plasma concentrations (average of 210±68.6 ng/mL) of nicotine were observed between 15 minutes and 1 hour post dosing. The average half-life after oral dosing was 0.949±0.214 hours. The average exposure for nicotine (Group 4) based on the dose normalized AUClast was 13.0±4.98 hr*kg*ng/mL/mg. On average, 3.31±0.91% and 0.09±0.07% of the dose (unchanged dose) was found in urine and feces, respectively, after PO dosing.

Following PO dosing of Reference Nicotine Polacrilex at 10 mg/kg (Group 4), maximum plasma concentrations (average of 14.3±4.74 ng/mL) of hydroxycotinine metabolite were observed between 45 minutes and 1 hour post dosing. The average half-life after oral dosing could not be determined. The average exposure for hydroxycotinine (Group 4) based on the dose normalized AUClast was 0.751±0.389 hr*kg*ng/mL/mg. On average, 6.48±2.12% and 0.03±0.02% of the dose was found in urine and feces, respectively, after PO dosing.

Following PO dosing of Reference Nicotine Polacrilex at 10 mg/kg (Group 4), maximum plasma concentrations (average of 223±71.9 ng/mL) of nicotine-n-oxide metabolite were observed between 15 minutes and 1 hour post dosing. The average half-life after oral dosing was 1.38 hours. The average exposure for nicotine-n-oxide (Group 4) based on the dose normalized AUClast was 15.0±6.27 hr*kg*ng/mL/mg. On average, 20.3±6.90% of the dose was found in urine after PO dosing. All concentrations in feces were below the limit of quantitation.

Following PO dosing of Reference Nicotine Polacrilex at 10 mg/kg (Group 4), maximum plasma concentrations (average of 247±49.4 ng/mL) of cotinine metabolite were observed between 45 minutes and 1 hour post dosing. The average half-life after oral dosing could not be determined. The average exposure for cotinine (Group 4) based on the dose normalized AUClast was 14.0±2.60 hr*kg*ng/mL/mg. On average, 5.30±2.18% and 0.16±0.08% of the dose was found in urine and feces, respectively, after PO dosing.

Following PO dosing of Reference Nicotine Polacrilex at 10 mg/kg (Groups 1-4), the average (±SE) C max for nicotine in brain tissue was 427±66.5 ng/g, the t max was 4 hours, the half-life could not be determined, and the exposure for nicotine based on the dose normalized AUClast was 588±53.8 hr*kg*ng/g/mg. After PO dosing of Reference Nicotine Polacrilex, the average (±SE) C max for hydroxycotinine metabolite in brain tissue was 51.8±9.14 ng/g, the t max was 8 hours, the half-life could not be determined, and the exposure for hydroxycotinine metabolite based on the dose normalized AUClast was 95.5±12.1 hr*kg*ng/g/mg. After PO dosing of Reference Nicotine Polacrilex, the majority of the concentrations were below the limit of quantitation and therefore, the pharmacokinetic parameters were not able to be calculated. After PO dosing of Reference Nicotine Polacrilex, the average (±SE) C max for cotinine metabolite in brain tissue was 722±135 ng/g, the t max was 8 hours, the half-life could not be determined, and the exposure for cotinine metabolite based on the dose normalized AUClast was 1332±208 hr*kg*ng/g/mg.

Following PO dosing of Reference Nicotine Polacrilex at 10 mg/kg (Groups 1-4), the average (±SE) C max for nicotine in liver tissue was 1300±308 ng/g, the t max was 4 hours, the half-life could not be determined, and the exposure for nicotine based on the dose normalized AUClast was 1737±167 hr*kg*ng/g/mg. After PO dosing of Reference Nicotine Polacrilex, the average (±SE) C max for hydroxycotinine metabolite in liver tissue was 102±13.5 ng/g, the t max was 8 hours, the half-life could not be determined, and the exposure for hydroxycotinine metabolite based on the dose normalized AUClast was 205±26.3 hr*kg*ng/g/mg. After PO dosing of Reference Nicotine Polacrilex, the average (±SE) C max for nicotine-n-oxide metabolite in liver tissue was 4.51±1.58 ng/g, the t max was 8 hours, the half-life could not be determined, and the exposure for nicotine-n-oxide metabolite based on the dose normalized AUClast was 6.86±1.83 hr*kg*ng/g/mg. After PO dosing of Reference Nicotine Polacrilex, the average (±SE) C max for cotinine metabolite in liver tissue was 905±119 ng/g, the t max was 8 hours, the half-life could not be determined, and the exposure for cotinine metabolite based on the dose normalized AUClast was 1620±189 hr*kg*ng/g/mg.

Following PO dosing of Reference Nicotine Polacrilex at 10 mg/kg (Groups 1-4), the average (±SE) C max for nicotine in kidney tissue was 8965±1519 ng/g, the t max was 4 hours, the half-life could not be determined, and the exposure for nicotine based on the dose normalized AUClast was 12267±1173 hr*kg*ng/g/mg. After PO dosing of Reference Nicotine Polacrilex, the average (±SE) C max for hydroxycotinine metabolite in kidney tissue was 200±44.1 ng/g, the t max was 24 hours, the half-life could not be determined, and the exposure for hydroxycotinine metabolite based on the dose normalized AUClast was 391±47.7 hr*kg*ng/g/mg. After PO dosing of Reference Nicotine Polacrilex, the average (±SE) C max for nicotine-n-oxide metabolite in kidney tissue was 20.5±4.26 ng/g, the t max was 4 hours, the half-life could not be determined, and the exposure for nicotine-n-oxide metabolite based on the dose normalized AUClast was 23.4±2.80 hr*kg*ng/g/mg. After PO dosing of Reference Nicotine Polacrilex, the average (±SE) C max for cotinine metabolite in kidney tissue was 1775±217 ng/g, the t max was 8 hours, the half-life could not be determined, and the exposure for cotinine metabolite based on the dose normalized AUClast was 3436±374 hr*kg*ng/g/mg.

Following PO dosing of Test Nicotine Polacrilex at 10 mg/kg (Group 5), maximum plasma concentrations (average of 416±255 ng/mL) of nicotine were observed between 12 minutes and 1 hour post dosing. The average half-life after oral dosing could not be determined. The average exposure for nicotine (Group 5) based on the dose normalized AUClast was 28.7±13.8 hr*kg*ng/mL/mg.

Following PO dosing of Test Nicotine Polacrilex at 10 mg/kg (Group 5), maximum plasma concentrations (average of 13.9±3.07 ng/mL) of hydroxycotinine metabolite were observed at 1 hour post dosing. The average half-life after oral dosing could not be determined. The average exposure for hydroxycotinine (Group 5) based on the dose normalized AUClast was 0.671±0.167 hr*kg*ng/mL/mg.

Following PO dosing of Test Nicotine Polacrilex at 10 mg/kg (Group 5), maximum plasma concentrations (average of 267±56.1 ng/mL) of nicotine-n-oxide metabolite were observed between 45 minutes and 1 hour post dosing. The average half-life after oral dosing could not be determined. The average exposure for nicotine-n-oxide (Group 5) based on the dose normalized AUClast was 19.3±3.45 hr*kg*ng/mL/mg.

Following PO dosing of Test Nicotine Polacrilex at 10 mg/kg (Group 5), maximum plasma concentrations (average of 381±81.8 ng/mL) of cotinine metabolite were observed at 1 hour post dosing. The average half-life after oral dosing could not be determined. The average exposure for cotinine (Group 5) based on the dose normalized AUClast was 21.3±5.76 hr*kg*ng/mL/mg.

Following PO dosing of Test Nicotine Polacrilex at 10 mg/kg (Group 6), maximum plasma concentrations (average of 315±142 ng/mL) of nicotine were observed between 15 minutes and 1 hour post dosing. The average half-life after oral dosing could not be determined. The average exposure for nicotine (Group 6) based on the dose normalized AUClast was 21.5±10.8 hr*kg*ng/mL/mg.

Following PO dosing of Test Nicotine Polacrilex at 10 mg/kg (Group 6), maximum plasma concentrations (average of 11.6±2.62 ng/mL) of hydroxycotinine metabolite were observed between 45 minutes and 1 hour post dosing. The average half-life after oral dosing could not be determined. The average exposure for hydroxycotinine (Group 6) based on the dose normalized AUClast was 0.581±0.149 hr*kg*ng/mL/mg.

Following PO dosing of Test Nicotine Polacrilex at 10 mg/kg (Group 6), maximum plasma concentrations (average of 246±120 ng/mL) of nicotine-n-oxide metabolite were observed between 15 minutes and 1 hour post dosing. The average half-life after oral dosing could not be determined. The average exposure for nicotine-n-oxide (Group 6) based on the dose normalized AUClast was 15.6±8.37 hr*kg*ng/mL/mg.

Following PO dosing of Test Nicotine Polacrilex at 10 mg/kg (Group 6), maximum plasma concentrations (average of 315±76.8 ng/mL) of cotinine metabolite were observed between 45 minutes and 1 hour post dosing. The average half-life after oral dosing could not be determined. The average exposure for cotinine (Group 6) based on the dose normalized AUClast was 17.7±5.25 hr*kg*ng/mL/mg.

Following PO dosing of Test Nicotine Polacrilex at 10 mg/kg (Group 7), maximum plasma concentrations (average of 253±40.0 ng/mL) of nicotine were observed between 12 minutes and 1 hour post dosing. The average half-life after oral dosing could not be determined. The average exposure for nicotine (Group 7) based on the dose normalized AUClast was 18.3±6.21 hr*kg*ng/mL/mg. On average, 2.02±1.21% and 0.04±0.04% of the dose (unchanged dose) was found in urine and feces, respectively, after PO dosing.

Following PO dosing of Test Nicotine Polacrilex at 10 mg/kg (Group 7), maximum plasma concentrations (average of 12.7±4.62 ng/mL) of hydroxycotinine metabolite were observed at 1 hour post dosing. The average half-life after oral dosing could not be determined. The average exposure for hydroxycotinine (Group 7) based on the dose normalized AUClast was 0.620±0.253 hr*kg*ng/mL/mg. On average, 0.97±0.34% and 0.02% (n=1) of the dose was found in urine and feces, respectively, after PO dosing.

Following PO dosing of Test Nicotine Polacrilex at 10 mg/kg (Group 7), maximum plasma concentrations (average of 276±67.5 ng/mL) of nicotine-n-oxide metabolite were observed between 15 minutes and 1 hour post dosing. The average half-life after oral dosing was 2.84 hours. The average exposure for nicotine-n-oxide (Group 7) based on the dose normalized AUClast was 17.6±6.17 hr*kg*ng/mL/mg. On average, 9.91±4.61% and 0.12% of the dose was found in urine and feces, respectively, after PO dosing.

Following PO dosing of Test Nicotine Polacrilex at 10 mg/kg (Group 7), maximum plasma concentrations (average of 317±100 ng/mL) of cotinine metabolite were observed at 1 hour post dosing. The average half-life after oral dosing could not be determined. The average exposure for cotinine (Group 7) based on the dose normalized AUClast was 16.6±4.69 hr*kg*ng/mL/mg. On average, 1.39±0.80% and 0.02±0.01% of the dose was found in urine and feces, respectively, after PO dosing.

Following PO dosing of Test Nicotine Polacrilex at 10 mg/kg (Group 8), maximum plasma concentrations (average of 593±641 ng/mL) of nicotine were observed between 8 minutes and 1 hour post dosing. The average half-life after oral dosing could not be determined; however, the half-life for one rat was 0.737 hours. The average exposure for nicotine (Group 8) based on the dose normalized AUClast was 38.0±38.5 hr*kg*ng/mL/mg. On average, 5.91±3.24% and 0.06±0.03% of the dose (unchanged dose) was found in urine and feces, respectively, after PO dosing.

Following PO dosing of Test Nicotine Polacrilex at 10 mg/kg (Group 8), maximum plasma concentrations (average of 17.4±13.8 ng/mL) of hydroxycotinine metabolite were observed between 45 minutes and 1 hour post dosing. The average half-life after oral dosing could not be determined. The average exposure for hydroxycotinine (Group 8) based on the dose normalized AUClast was 0.940±0.788 hr*kg*ng/mL/mg. On average, 9.07±3.61% and 0.02±0.01% of the dose was found in urine and feces, respectively, after PO dosing.

Following PO dosing of Test Nicotine Polacrilex at 10 mg/kg (Group 8), maximum plasma concentrations (average of 357±306 ng/mL) of nicotine-n-oxide metabolite were observed between 15 minutes and 1 hour post dosing. The average half-life after oral dosing could not be determined; however, the half-life for one rat was 0.888 hours. The average exposure for nicotine-n-oxide (Group 8) based on the dose normalized AUClast was 27.5±23.8 hr*kg*ng/mL/mg. On average, 39.5±9.71% and 0.08% of the dose was found in urine and feces, respectively, after PO dosing.

Following PO dosing of Test Nicotine Polacrilex at 10 mg/kg (Group 8), maximum plasma concentrations (average of 441±333 ng/mL) of cotinine metabolite were observed at 1 hour post dosing. The average half-life after oral dosing could not be determined. The average exposure for cotinine (Group 8) based on the dose normalized AUClast was 25.8±20.0 hr*kg*ng/mL/mg. On average, 8.23±2.58% and 0.18±0.10% of the dose was found in urine and feces, respectively, after PO dosing.

Following PO dosing of Test Nicotine Polacrilex at 10 mg/kg (Groups 5-8), the average (±SE) C max for nicotine in brain tissue was 1260±200 ng/g, the t max was 1 hour, the half-life was 21.6 hours, and the exposure for nicotine based on the dose normalized AUClast was 1300±125 hr*kg*ng/g/mg. After PO dosing of Test Nicotine Polacrilex, the average (±SE) C max for hydroxycotinine metabolite in brain tissue was 91.2±7.69 ng/g, the t max was 24 hours, the half-life could not be determined, and the exposure for hydroxycotinine metabolite based on the dose normalized AUClast was 142±6.64 hr*kg*ng/g/mg. After PO dosing of Test Nicotine Polacrilex, the average (±SE) C max for nicotine-n-oxide metabolite in brain tissue was 4.17±1.41 ng/g, the t max was 1 hour, the half-life could not be determined, and the exposure for nicotine-n-oxide metabolite based on the dose normalized AUClast was 2.70±1.05 hr*kg*ng/g/mg. After PO dosing of Test Nicotine Polacrilex, the average (±SE) C max for cotinine metabolite in brain tissue was 1322±219 ng/g, the t max was 24 hours, the half-life could not be determined, and the exposure for cotinine metabolite based on the dose normalized AUClast was 2172±189 hr*kg*ng/g/mg.

Following PO dosing of Test Nicotine Polacrilex at 10 mg/kg (Groups 5-8), the average (±SE) C max for nicotine in liver tissue was 2702±308 ng/g, the t max was 1 hour, the half-life was 18.9 hours, and the exposure for nicotine based on the dose normalized AUClast was 2989±277 hr*kg*ng/g/mg. After PO dosing of Test Nicotine Polacrilex, the average (±SE) C max for hydroxycotinine metabolite in liver tissue was 232±41.2 ng/g, the t max was 24 hours, the half-life could not be determined, and the exposure for hydroxycotinine metabolite based on the dose normalized AUClast was 338±37.6 hr*kg*ng/g/mg. After PO dosing of Test Nicotine Polacrilex, the average (±SE) C max for nicotine-n-oxide metabolite in liver tissue was 6.69±1.67 ng/g, the t max was 1 hour, the half-life could not be determined, and the exposure for nicotine-n-oxide metabolite based on the dose normalized AUClast was 8.74±2.56 hr*kg*ng/g/mg. After PO dosing of Test Nicotine Polacrilex, the average (±SE) C max for cotinine metabolite in liver tissue was 1451±157 ng/g, the t max was 24 hours, the half-life could not be determined, and the exposure for cotinine metabolite based on the dose normalized AUClast was 2505±139 hr*kg*ng/g/mg.

Following PO dosing of Test Nicotine Polacrilex at 10 mg/kg (Groups 5-8), the average (±SE) C max for nicotine in kidney tissue was 8930±676 ng/g, the t max was 1 hour, the half-life was 24.2 hours, and the exposure for nicotine based on the dose normalized AUClast was 12717±1354 hr*kg*ng/g/mg. After PO dosing of Test Nicotine Polacrilex, the average (±SE) C max for hydroxycotinine metabolite in kidney tissue was 244±16.5 ng/g, the t max was 24 hours, the half-life could not be determined, and the exposure for hydroxycotinine metabolite based on the dose normalized AUClast was 449±24.1 hr*kg*ng/g/mg. After PO dosing of Test Nicotine Polacrilex, the average (±SE) C max for nicotine-n-oxide metabolite in kidney tissue was 28.0±6.34 ng/g, the t max was 1 hour, the half-life could not be determined, and the exposure for nicotine-n-oxide metabolite based on the dose normalized AUClast was 38.0±5.57 hr*kg*ng/g/mg. After PO dosing of Test Nicotine Polacrilex, the average (±SE) C max for cotinine metabolite in kidney tissue was 2466±321 ng/g, the t max was 24 hours, the half-life could not be determined, and the exposure for cotinine metabolite based on the dose normalized AUClast was 4300±280 hr*kg*ng/g/mg.

### Example 6

In this study, the exposure and distribution of nicotine and its major metabolites were evaluated following oral administration of two separate formulations (Reference and Test Nicotine Polacrilex) in male Sprague-Dawley rats. Formulations were administered orally (PO) at 10 mg/kg. Following dosing, blood samples were collected up to 1 hour post dose; and urine and fecal samples were collected up to 24 hours post dose. Brain, liver, and kidney tissue were collected at 1 hour (Groups 1 & 5), 4 hours (Groups 2 & 6), following the 8 hour urine and feces sample collection (Groups 3 & 7), or following the 24 hour urine and feces sample collection (Groups 4 & 8). Blood, urine, feces, and tissue concentrations of each analyte were determined by LC-MS/MS. Plasma pharmacokinetic parameters were determined using WinNonlin (v8.0). Brain, liver, and kidney pharmacokinetic parameters were determined using WinNonlin (v8.0) software with sparse sampling.

Following PO dosing of Reference Nicotine Polacrilex at 10 mg/kg (Group 1), maximum plasma concentrations (average of 144±68.2 ng/mL) of nicotine were observed between 30 minutes and 1 hour post dosing. The average half-life after oral dosing could not be determined. The average exposure for nicotine (Group 1) based on the dose normalized AUClast was 8.71±2.76 hr*kg*ng/mL/mg.

Following PO dosing of Reference Nicotine Polacrilex at 10 mg/kg (Group 1), maximum plasma concentrations (average of 9.79±3.56 ng/mL) of hydroxycotinine metabolite were observed between 45 minutes and 1 hour post dosing. The average half-life after oral dosing could not be determined. The average exposure for hydroxycotinine (Group 1) based on the dose normalized AUClast was 0.420±0.146 hr*kg*ng/mL/mg.

Following PO dosing of Reference Nicotine Polacrilex at 10 mg/kg (Group 1), maximum plasma concentrations (average of 179±54.9 ng/mL) of nicotine-n-oxide metabolite were observed between 30 minutes and 1 hour post dosing. The average half-life after oral dosing could not be determined. The average exposure for nicotine-n-oxide (Group 1) based on the dose normalized AUClast was 11.2±3.32 hr*kg*ng/mL/mg.

Following PO dosing of Reference Nicotine Polacrilex at 10 mg/kg (Group 1), maximum plasma concentrations (average of 193±58.6 ng/mL) of cotinine metabolite were observed at 1 hour post dosing. The average half-life after oral dosing could not be determined. The average exposure for cotinine (Group 1) based on the dose normalized AUClast was 10.9±2.90 hr*kg*ng/mL/mg.

Following PO dosing of Reference Nicotine Polacrilex at 10 mg/kg (Group 2), maximum plasma concentrations (average of 350±256 ng/mL) of nicotine were observed between 8 minutes and 1 hour post dosing. The average half-life after oral dosing could not be determined. The average exposure for nicotine (Group 2) based on the dose normalized AUClast was 21.3±13.7 hr*kg*ng/mL/mg.

Following PO dosing of Reference Nicotine Polacrilex at 10 mg/kg (Group 2), maximum plasma concentrations (average of 20.1±13.3 ng/mL) of hydroxycotinine metabolite were observed at 1 hour post dosing. The average half-life after oral dosing could not be determined. The average exposure for hydroxycotinine (Group 2) based on the dose normalized AUClast was 1.15±0.928 hr*kg*ng/mL/mg.

Following PO dosing of Reference Nicotine Polacrilex at 10 mg/kg (Group 2), maximum plasma concentrations (average of 409±235 ng/mL) of nicotine-n-oxide metabolite were observed between 12 minutes and 1 hour post dosing. The average half-life after oral dosing could not be determined. The average exposure for nicotine-n-oxide (Group 2) based on the dose normalized AUClast was 26.8±18.3 hr*kg*ng/mL/mg.

Following PO dosing of Reference Nicotine Polacrilex at 10 mg/kg (Group 2), maximum plasma concentrations (average of 359±236 ng/mL) of cotinine metabolite were observed between 45 minutes and 1 hour post dosing. The average half-life after oral dosing could not be determined. The average exposure for cotinine (Group 2) based on the dose normalized AUClast was 22.5±16.7 hr*kg*ng/mL/mg.

Following PO dosing of Reference Nicotine Polacrilex at 10 mg/kg (Group 3), maximum plasma concentrations (average of 176±71.2 ng/mL) of nicotine were observed between 30 minutes and 1 hour post dosing. The average half-life after oral dosing could not be determined. The average exposure for nicotine (Group 3) based on the dose normalized AUClast was 11.7±4.62 hr*kg*ng/mL/mg. On average, 1.04±0.49% and 0.03±0.04% of the dose (unchanged dose) was found in urine and feces, respectively, after PO dosing.

Following PO dosing of Reference Nicotine Polacrilex at 10 mg/kg (Group 3), maximum plasma concentrations (average of 13.4±5.95 ng/mL) of hydroxycotinine metabolite were observed between 45 minutes 1 hour post dosing. The average half-life after oral dosing could not be determined. The average exposure for hydroxycotinine (Group 3) based on the dose normalized AUClast was 0.672±0.386 hr*kg*ng/mL/mg. On average, 1.10±0.64% and 0.03% (n=1) of the dose was found in urine and feces, respectively, after PO dosing.

Following PO dosing of Reference Nicotine Polacrilex at 10 mg/kg (Group 3), maximum plasma concentrations (average of 283±134 ng/mL) of nicotine-n-oxide metabolite were observed between 30 minutes and 1 hour post dosing. The average half-life after oral dosing could not be determined. The average exposure for nicotine-n-oxide (Group 3) based on the dose normalized AUClast was 17.8±7.29 hr*kg*ng/mL/mg. On average, 9.36±4.36% and 0.07% (n=1) of the dose was found in urine and feces, respectively, after PO dosing.

Following PO dosing of Reference Nicotine Polacrilex at 10 mg/kg (Group 3), maximum plasma concentrations (average of 304±103 ng/mL) of cotinine metabolite were observed at 1 hour post dosing. The average half-life after oral dosing could not be determined. The average exposure for cotinine (Group 3) based on the dose normalized AUClast was 15.4±4.99 hr*kg*ng/mL/mg. On average, 0.99±0.48% and 0.03±0.02% of the dose was found in urine and feces, respectively, after PO dosing.

Following PO dosing of Reference Nicotine Polacrilex at 10 mg/kg (Group 4), maximum plasma concentrations (average of 210±68.6 ng/mL) of nicotine were observed between 15 minutes and 1 hour post dosing. The average half-life after oral dosing was 0.949±0.214 hours. The average exposure for nicotine (Group 4) based on the dose normalized AUClast was 13.0±4.98 hr*kg*ng/mL/mg. On average, 3.31±0.91% and 0.09±0.07% of the dose (unchanged dose) was found in urine and feces, respectively, after PO dosing.

Following PO dosing of Reference Nicotine Polacrilex at 10 mg/kg (Group 4), maximum plasma concentrations (average of 14.3±4.74 ng/mL) of hydroxycotinine metabolite were observed between 45 minutes and 1 hour post dosing. The average half-life after oral dosing could not be determined. The average exposure for hydroxycotinine (Group 4) based on the dose normalized AUClast was 0.751±0.389 hr*kg*ng/mL/mg. On average, 6.48±2.12% and 0.03±0.02% of the dose was found in urine and feces, respectively, after PO dosing.

Following PO dosing of Reference Nicotine Polacrilex at 10 mg/kg (Group 4), maximum plasma concentrations (average of 223±71.9 ng/mL) of nicotine-n-oxide metabolite were observed between 15 minutes and 1 hour post dosing. The average half-life after oral dosing was 1.38 hours. The average exposure for nicotine-n-oxide (Group 4) based on the dose normalized AUClast was 15.0±6.27 hr*kg*ng/mL/mg. On average, 20.3±6.90% of the dose was found in urine after PO dosing. All concentrations in feces were below the limit of quantitation.

Following PO dosing of Reference Nicotine Polacrilex at 10 mg/kg (Group 4), maximum plasma concentrations (average of 247±49.4 ng/mL) of cotinine metabolite were observed between 45 minutes and 1 hour post dosing. The average half-life after oral dosing could not be determined. The average exposure for cotinine (Group 4) based on the dose normalized AUClast was 14.0±2.60 hr*kg*ng/mL/mg. On average, 5.30±2.18% and 0.16±0.08% of the dose was found in urine and feces, respectively, after PO dosing.

Following PO dosing of Reference Nicotine Polacrilex at 10 mg/kg (Groups 1-4), the average (±SE) C max for nicotine in brain tissue was 427±66.5 ng/g, the t max was 4 hours, the half-life could not be determined, and the exposure for nicotine based on the dose normalized AUClast was 588±53.8 hr*kg*ng/g/mg. After PO dosing of Reference Nicotine Polacrilex, the average (±SE) C max for hydroxycotinine metabolite in brain tissue was 51.8±9.14 ng/g, the t max was 8 hours, the half-life could not be determined, and the exposure for hydroxycotinine metabolite based on the dose normalized AUClast was 95.5±12.1 hr*kg*ng/g/mg. After PO dosing of Reference Nicotine Polacrilex, the majority of the concentrations were below the limit of quantitation and therefore, the pharmacokinetic parameters were not able to be calculated. After PO dosing of Reference Nicotine Polacrilex, the average (±SE) C max for cotinine metabolite in brain tissue was 722±135 ng/g, the t max was 8 hours, the half-life could not be determined, and the exposure for cotinine metabolite based on the dose normalized AUClast was 1332±208 hr*kg*ng/g/mg.

Following PO dosing of Reference Nicotine Polacrilex at 10 mg/kg (Groups 1-4), the average (±SE) C max for nicotine in liver tissue was 1300±308 ng/g, the t max was 4 hours, the half-life could not be determined, and the exposure for nicotine based on the dose normalized AUClast was 1737±167 hr*kg*ng/g/mg. After PO dosing of Reference Nicotine Polacrilex, the average (±SE) C max for hydroxycotinine metabolite in liver tissue was 102±13.5 ng/g, the t max was 8 hours, the half-life could not be determined, and the exposure for hydroxycotinine metabolite based on the dose normalized AUClast was 205±26.3 hr*kg*ng/g/mg. After PO dosing of Reference Nicotine Polacrilex, the average (±SE) C max for nicotine-n-oxide metabolite in liver tissue was 4.51±1.58 ng/g, the t max was 8 hours, the half-life could not be determined, and the exposure for nicotine-n-oxide metabolite based on the dose normalized AUClast was 6.86±1.83 hr*kg*ng/g/mg. After PO dosing of Reference Nicotine Polacrilex, the average (±SE) C max for cotinine metabolite in liver tissue was 905±119 ng/g, the t max was 8 hours, the half-life could not be determined, and the exposure for cotinine metabolite based on the dose normalized AUClast was 1620±189 hr*kg*ng/g/mg.

Following PO dosing of Reference Nicotine Polacrilex at 10 mg/kg (Groups 1-4), the average (±SE) C max for nicotine in kidney tissue was 8965±1519 ng/g, the t max was 4 hours, the half-life could not be determined, and the exposure for nicotine based on the dose normalized AUClast was 12267±1173 hr*kg*ng/g/mg. After PO dosing of Reference Nicotine Polacrilex, the average (±SE) C max for hydroxycotinine metabolite in kidney tissue was 200±44.1 ng/g, the t max was 24 hours, the half-life could not be determined, and the exposure for hydroxycotinine metabolite based on the dose normalized AUClast was 391±47.7 hr*kg*ng/g/mg. After PO dosing of Reference Nicotine Polacrilex, the average (±SE) C max for nicotine-n-oxide metabolite in kidney tissue was 20.5±4.26 ng/g, the t max was 4 hours, the half-life could not be determined, and the exposure for nicotine-n-oxide metabolite based on the dose normalized AUClast was 23.4±2.80 hr*kg*ng/g/mg. After PO dosing of Reference Nicotine Polacrilex, the average (±SE) C max for cotinine metabolite in kidney tissue was 1775±217 ng/g, the t max was 8 hours, the half life could not be determined, and the exposure for cotinine metabolite based on the dose normalized AUClast was 3436±374 hr*kg*ng/g/mg.

Following PO dosing of Test Nicotine Polacrilex at 10 mg/kg (Group 5), maximum plasma concentrations (average of 416±255 ng/mL) of nicotine were observed between 12 minutes and 1 hour post dosing. The average half-life after oral dosing could not be determined. The average exposure for nicotine (Group 5) based on the dose normalized AUClast was 28.7±13.8 Following PO dosing of Test Nicotine Polacrilex at 10 mg/kg (Group 5), maximum plasma concentrations (average of 13.9±3.07 ng/mL) of hydroxycotinine metabolite were observed at 1 hour post dosing. The average half-life after oral dosing could not be determined. The average exposure for hydroxycotinine (Group 5) based on the dose normalized AUClast was 0.671±0.167 hr*kg*ng/mL/mg.

Following PO dosing of Test Nicotine Polacrilex at 10 mg/kg (Group 5), maximum plasma concentrations (average of 267±56.1 ng/mL) of nicotine-n-oxide metabolite were observed between 45 minutes and 1 hour post dosing. The average half-life after oral dosing could not be determined. The average exposure for nicotine-n-oxide (Group 5) based on the dose normalized AUClast was 19.3±3.45 hr*kg*ng/mL/mg.

Following PO dosing of Test Nicotine Polacrilex at 10 mg/kg (Group 5), maximum plasma concentrations (average of 381±81.8 ng/mL) of cotinine metabolite were observed at 1 hour post dosing. The average half-life after oral dosing could not be determined. The average exposure for cotinine (Group 5) based on the dose normalized AUClast was 21.3±5.76 hr*kg*ng/mL/mg.

Following PO dosing of Test Nicotine Polacrilex at 10 mg/kg (Group 6), maximum plasma concentrations (average of 315±142 ng/mL) of nicotine were observed between 15 minutes and 1 hour post dosing. The average half-life after oral dosing could not be determined. The average exposure for nicotine (Group 6) based on the dose normalized AUClast was 21.5±10.8 hr*kg*ng/mL/mg.

Following PO dosing of Test Nicotine Polacrilex at 10 mg/kg (Group 6), maximum plasma concentrations (average of 11.6±2.62 ng/mL) of hydroxycotinine metabolite were observed between 45 minutes and 1 hour post dosing. The average half-life after oral dosing could not be determined. The average exposure for hydroxycotinine (Group 6) based on the dose normalized AUClast was 0.581±0.149 hr*kg*ng/mL/mg.

Following PO dosing of Test Nicotine Polacrilex at 10 mg/kg (Group 6), maximum plasma concentrations (average of 246±120 ng/mL) of nicotine-n-oxide metabolite were observed between 15 minutes and 1 hour post dosing. The average half-life after oral dosing could not be determined. The average exposure for nicotine-n-oxide (Group 6) based on the dose normalized AUClast was 15.6±8.37 hr*kg*ng/mL/mg.

Following PO dosing of Test Nicotine Polacrilex at 10 mg/kg (Group 6), maximum plasma concentrations (average of 315±76.8 ng/mL) of cotinine metabolite were observed between 45 minutes and 1 hour post dosing. The average half-life after oral dosing could not be determined. The average exposure for cotinine (Group 6) based on the dose normalized AUClast was 17.7±5.25 hr*kg*ng/mL/mg. Following PO dosing of Test Nicotine Polacrilex at 10 mg/kg (Group 7), maximum plasma concentrations (average of 253±40.0 ng/mL) of nicotine were observed between 12 minutes and 1 hour post dosing. The average half-life after oral dosing could not be determined. The average exposure for nicotine (Group 7) based on the dose normalized AUClast was 18.3±6.21 hr*kg*ng/mL/mg. On average, 2.02±1.21% and 0.04±0.04% of the dose (unchanged dose) was found in urine and feces, respectively, after PO dosing.

Following PO dosing of Test Nicotine Polacrilex at 10 mg/kg (Group 7), maximum plasma concentrations (average of 12.7±4.62 ng/mL) of hydroxycotinine metabolite were observed at 1 hour post dosing. The average half-life after oral dosing could not be determined. The average exposure for hydroxycotinine (Group 7) based on the dose normalized AUClast was 0.620±0.253 hr*kg*ng/mL/mg. On average, 0.97±0.34% and 0.02% (n=1) of the dose was found in urine and feces, respectively, after PO dosing.

Following PO dosing of Test Nicotine Polacrilex at 10 mg/kg (Group 7), maximum plasma concentrations (average of 276±67.5 ng/mL) of nicotine-n-oxide metabolite were observed between 15 minutes and 1 hour post dosing. The average half-life after oral dosing was 2.84 hours. The average exposure for nicotine-n-oxide (Group 7) based on the dose normalized AUClast was 17.6±6.17 hr*kg*ng/mL/mg. On average, 9.91±4.61% and 0.12% of the dose was found in urine and feces, respectively, after PO dosing.

Following PO dosing of Test Nicotine Polacrilex at 10 mg/kg (Group 7), maximum plasma concentrations (average of 317±100 ng/mL) of cotinine metabolite were observed at 1 hour post dosing. The average half-life after oral dosing could not be determined. The average exposure for cotinine (Group 7) based on the dose normalized AUClast was 16.6±4.69 hr*kg*ng/mL/mg. On average, 1.39±0.80% and 0.02±0.01% of the dose was found in urine and feces, respectively, after PO dosing.

Following PO dosing of Test Nicotine Polacrilex at 10 mg/kg (Group 8), maximum plasma concentrations (average of 593±641 ng/mL) of nicotine were observed between 8 minutes and 1 hour post dosing. The average half-life after oral dosing could not be determined; however, the half-life for one rat was 0.737 hours. The average exposure for nicotine (Group 8) based on the dose normalized AUClast was 38.0±38.5 hr*kg*ng/mL/mg. On average, 5.91±3.24% and 0.06±0.03% of the dose (unchanged dose) was found in urine and feces, respectively, after PO dosing.

Following PO dosing of Test Nicotine Polacrilex at 10 mg/kg (Group 8), maximum plasma concentrations (average of 17.4±13.8 ng/mL) of hydroxycotinine metabolite were observed between 45 minutes and 1 hour post dosing. The average half-life after oral dosing could not be determined. The average exposure for hydroxycotinine (Group 8) based on the dose normalized

AUClast was 0.940±0.788 hr*kg*ng/mL/mg. On average, 9.07±3.61% and 0.02±0.01% of the dose was found in urine and feces, respectively, after PO dosing.

Following PO dosing of Test Nicotine Polacrilex at 10 mg/kg (Group 8), maximum plasma concentrations (average of 357±306 ng/mL) of nicotine-n-oxide metabolite were observed between 15 minutes and 1 hour post dosing. The average half-life after oral dosing could not be determined; however, the half-life for one rat was 0.888 hours. The average exposure for nicotine-n-oxide (Group 8) based on the dose normalized AUClast was 27.5±23.8 hr*kg*ng/mL/mg. On average, 39.5±9.71% and 0.08% of the dose was found in urine and feces, respectively, after PO dosing.

Following PO dosing of Test Nicotine Polacrilex at 10 mg/kg (Group 8), maximum plasma concentrations (average of 441±333 ng/mL) of cotinine metabolite were observed at 1 hour post dosing. The average half-life after oral dosing could not be determined. The average exposure for cotinine (Group 8) based on the dose normalized AUClast was 25.8±20.0 hr*kg*ng/mL/mg. On average, 8.23±2.58% and 0.18±0.10% of the dose was found in urine and feces, respectively, after PO dosing.

Following PO dosing of Test Nicotine Polacrilex at 10 mg/kg (Groups 5-8), the average (±SE) C max for nicotine in brain tissue was 1260±200 ng/g, the t max was 1 hour, the half-life was 21.6 hours, and the exposure for nicotine based on the dose normalized AUClast was 1300±125 hr*kg*ng/g/mg. After PO dosing of Test Nicotine Polacrilex, the average (±SE) C max for hydroxycotinine metabolite in brain tissue was 91.2±7.69 ng/g, the t max was 24 hours, the half life could not be determined, and the exposure for hydroxycotinine metabolite based on the dose normalized AUClast was 142±6.64 hr*kg*ng/g/mg. After PO dosing of Test Nicotine Polacrilex, the average (±SE) C max for nicotine-n-oxide metabolite in brain tissue was 4.17±1.41 ng/g, the t max was 1 hour, the half-life could not be determined, and the exposure for nicotine-n-oxide metabolite based on the dose normalized AUClast was 2.70±1.05 hr*kg*ng/g/mg. After PO dosing of Test Nicotine Polacrilex, the average (±SE) C max for cotinine metabolite in brain tissue was 1322±219 ng/g, the t max was 24 hours, the half-life could not be determined, and the exposure for cotinine metabolite based on the dose normalized AUClast was 2172±189 hr*kg*ng/g/mg.

Following PO dosing of Test Nicotine Polacrilex at 10 mg/kg (Groups 5-8), the average (±SE) C max for nicotine in liver tissue was 2702±308 ng/g, the t max was 1 hour, the half-life was 18.9 hours, and the exposure for nicotine based on the dose normalized AUClast was 2989±277 hr*kg*ng/g/mg. After PO dosing of Test Nicotine Polacrilex, the average (±SE) C max for hydroxycotinine metabolite in liver tissue was 232±41.2 ng/g, the t max was 24 hours, the half life could not be determined, and the exposure for hydroxycotinine metabolite based on the dose normalized AUClast was 338±37.6 hr*kg*ng/g/mg. After PO dosing of Test Nicotine Polacrilex, the average (±SE) C max for nicotine-n-oxide metabolite in liver tissue was 6.69±1.67 ng/g, the t max was 1 hour, the half-life could not be determined, and the exposure for nicotine-n-oxide metabolite based on the dose normalized AUClast was 8.74±2.56 hr*kg*ng/g/mg. After PO dosing of Test Nicotine Polacrilex, the average (±SE) C max for cotinine metabolite in liver tissue was 1451±157 ng/g, the t max was 24 hours, the half-life could not be determined, and the exposure for cotinine metabolite based on the dose normalized AUClast was 2505±139 hr*kg*ng/g/mg.

Following PO dosing of Test Nicotine Polacrilex at 10 mg/kg (Groups 5-8), the average (±SE) C max for nicotine in kidney tissue was 8930±676 ng/g, the t max was 1 hour, the half-life was 24.2 hours, and the exposure for nicotine based on the dose normalized AUClast was 12717±1354 hr*kg*ng/g/mg. After PO dosing of Test Nicotine Polacrilex, the average (±SE) C max for hydroxycotinine metabolite in kidney tissue was 244±16.5 ng/g, the t max was 24 hours, the half life could not be determined, and the exposure for hydroxycotinine metabolite based on the dose normalized AUClast was 449±24.1 hr*kg*ng/g/mg. After PO dosing of Test Nicotine Polacrilex, the average (±SE) C max for nicotine-n-oxide metabolite in kidney tissue was 28.0±6.34 ng/g, the t max was 1 hour, the half-life could not be determined, and the exposure for nicotine-n-oxide metabolite based on the dose normalized AUClast was 38.0±5.57 hr*kg*ng/g/mg. After PO dosing of Test Nicotine Polacrilex, the average (±SE) C max for cotinine metabolite in kidney tissue was 2466±321 ng/g, the t max was 24 hours, the half-life could not be determined, and the exposure for cotinine metabolite based on the dose normalized AUClast was 4300±280 hr*kg*ng/g/mg.

### Example 7

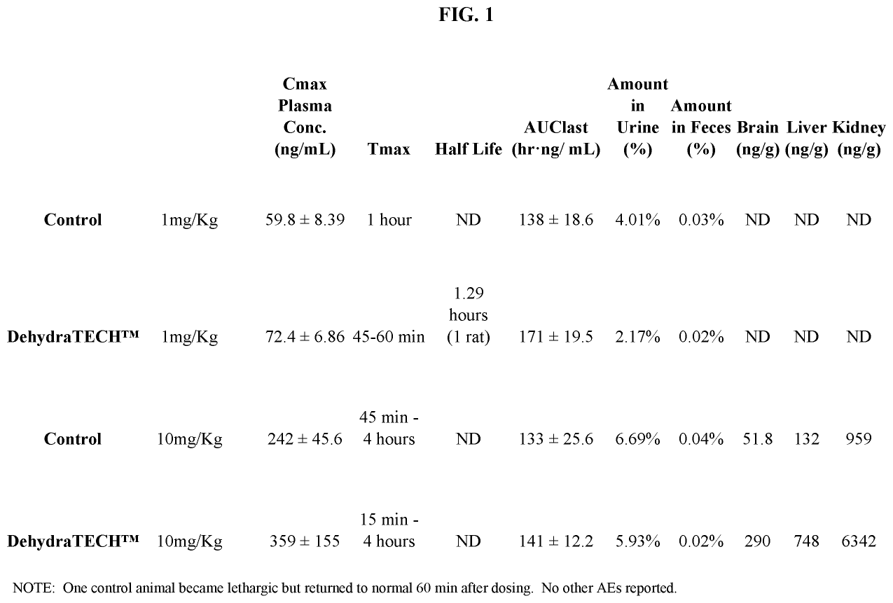

Pharmacokinetic (PK) results from Examples 5 and 6 were compared. FIG. 1 shows PK results from Example 5 comparing nicotine concentrations in various tissues following administration of DEHYDRATECH™ and control compositions in rats.

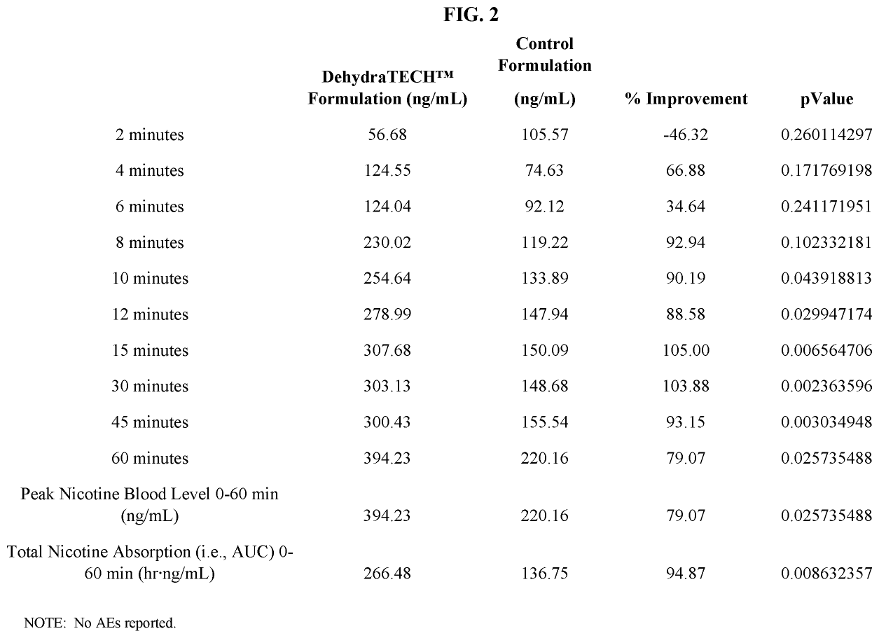

FIG. 2 shows results from Example 6 showing improvement in peak nicotine blood levels following administration of DEHYDRATECH™ and control compositions in rats. A significant improvement in the DEHYDRATECH™ compared to control formulation was observed by 10 minutes after administration.

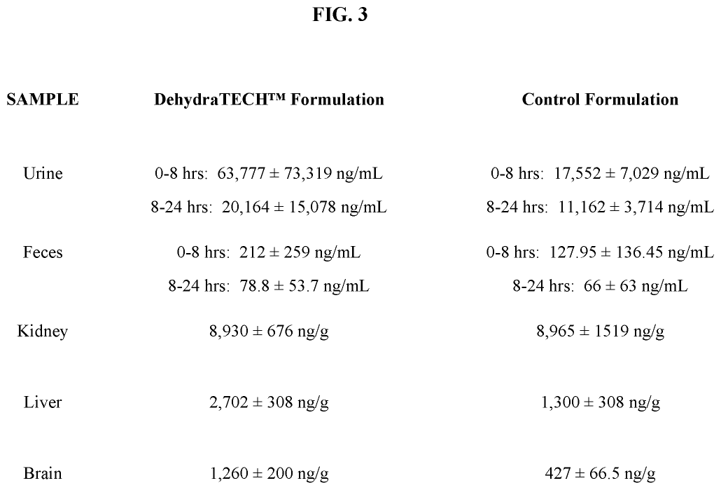

FIG. 3 shows results from Example 6 comparing nicotine concentrations in various tissues following administration of DEHYDRATECH™ and control compositions in rats. A significantly greater concentration of nicotine was observed in brain tissue in the DEHYDRATECH™ treated animals compared to the control formulation.

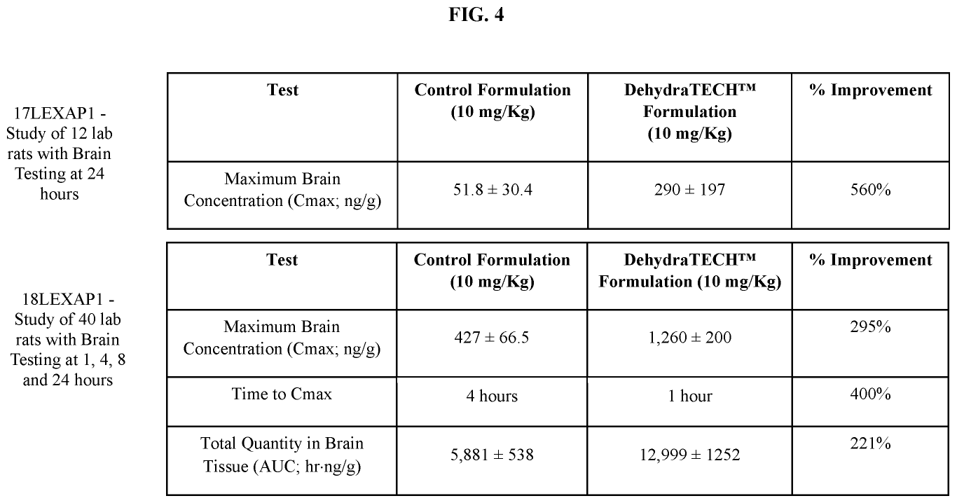

FIG. 4 shows results from Examples 5 and 6 comparing improvements in maximum brain concentration, time to C max, and total quantity in brain tissue following administration of DEHYDRATECH™ and control compositions in rats at various time points. Improvement by orders of magnitude were observed in the DEHYDRATECH™ compared to control formulations.

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

1.-65. (canceled)

66. A composition, comprising:
a) a therapeutically effective amount of nicotine, a nicotine derivative, or a salt thereof;
b) a bioavailability enhancing agent comprising an edible oil; and
c) an edible substrate.

67. The composition according to claim 66, wherein the composition comprises nicotine.

68. The composition according to claim 66, wherein the nicotine derivative is selected from the group consisting of nicotine bitartrate, lobeline, cytisine, nicotine polacrilex, nornicotine, nicotine 1-N-oxide, metanicotine, nicotine imine, nicotine N-glucuronide, N-methylnicotinium, N-n-decylnicotinium, 5′-cyanonicotine, 3,4-dihydrometanicotine, N′-methylnicotinium, N-octanoylnornicotine, 2,3,3a,4,5,9b-hexahydro-1-methyl-1H-pyrrolo(3,2-h)isoquinoline, 5-isothiocyanonicotine, 5-iodonicotine, 5′-hydroxycotinine-N-oxide, homoazanicotine, nicotine monomethiodide, N-4-azido-2-nitrophenylnornicotine, N-methylnornicotinium, nicotinium molybdophosphate resin, N-methyl-N′-oxonicotinium, N′-propylnornicotine, pseudooxynicotine, 4′-methylnicotine, 5-fluoronicotine, K(s-nic)5(Ga2(N,N′-bis-(2,3-dihydroxybenzoyl)-1,4-phenylenediamine)3), 5-methoxynicotine, 1-benzyl-4-phenylnicotinamidinium, 6-n-propylnicotine, SIB1663, 6-hydroxynicotine, N-methyl-nicotine, 6-(2-phenylethyl)nicotine, N′-formylnornicotine, N-n-octylnicotinium, N-(n-oct-3-enyl)nicotinium, N-(n-dec-9-enyl)nicotinium, 5′-acetoxy-N′-nitrosonornicotine, 4-hydroxynicotine, 4-(dimethylphenylsilyl)nicotine, N′-carbomethoxynornicotine, and N-methylnicoton.

69. The composition according to claim 66, comprising nicotine hydrogen tartrate, nicotine bitartrate dihydrate, nicotine hydrochloride, nicotine dihydrochloride, nicotine sulfate, nicotine citrate, nicotine zinc chloride monohydrate, nicotine salicylate, nicotine oil, or nicotine complexed with cyclodextrin.

70. The composition according to claim 66, wherein the nicotine derivative is an agonist having selectivity to an α₇ nicotinic receptor subtype, wherein the α₇ nicotinic receptor subtype agonist is selected from the group consisting of N-[(2S,3S)-2-(pyridin-3-ylmethyl)-1-azabicyclo[2.2.2]oct-3-yl]-1-benzofuran-2-carboxamide, (5aS,8S,10aR)-5a,6,9,10-Tetrahydro,7H,11H-8,10a-methanopyrido[2′,3′:5,6]pyrano[2,3-d]azepine, 1,4-diazabicyclo[3.2.2]nonane-4-carboxylic acid, 4-bromophenyl ester, 3-[(3E)-3-[(2,4-dimethoxyphenyl)methylidene]-5,6-dihydro-4H-pyridin-2-yl]-pyridine, 2-methyl-5-(6-phenyl-pyridazin-3-yl)-octahydro-pyrrolo[3,4-c]pyrrole, (5 S)-spiro[1,3-oxazolidine-5,8′-1-azabicyclo[2.2.2]octane]-2-one, N-[(3R)-1-azabicyclo[2.2.2]oct-3-yl]-4-chlorobenzamide, 5-morpholin-4-yl-pentanoic acid (4-pyridin-3-yl-phenyl)-amide, EVP-6124, EVP-4473, TC-6987, and MEM3454, or the nicotine derivative is an agonist having selectivity to an α₄β₂ nicotinic receptor subtype, wherein the α₄β₂ nicotinic receptor subtype agonist is selected from the group consisting of 7,8,9,10-tetrahydro-6,10-methano-6H-pyrazino(2,3-h)(3) benzazepine, (2S,4E)-5-(5-isopropoxypyridin-3-yl)-N-methylpent-4-en-2-amine, [3-(2(S))-azetidinylmethoxy)71 pyridine] dihydrochloride, (5aS,8S,10aR)-5a,6,9,10-Tetrahydro,7H,11H-8,10a-methanopyrido [2′,3′:5,6]pyrano[2,3-d]azepine, A-969933, S35836-1, S35678-1, and 3-(5,6-Dichloro-pyridin-3-yl)-1S,5S-3,6-diazabicyclo[3.2.0]heptane.

71. The composition according to claim 66, wherein the bioavailability enhancing agent bioavailability enhancing agent is a protective colloid, an edible oil or fat.

72. The composition according to claim 71, wherein the edible oil is coconut oil, peanut oil, soybean oil, safflower seed oil, corn oil, olive oil, castor oil, cottonseed oil, arachis oil, or sunflower seed oil.

73. The composition according to claim 72, wherein the edible oil comprises fatty acids selected from the group consisting of oleic acid, undecanoic acid, valeric acid, heptanoic acid, pelargonic acid, capric acid, lauric acid, and eicosapentaenoic acid.

74. The composition according to claim 66, wherein the edible substrate is selected from the group consisting of inulin, starch, modified starches, xanthan gum, carboxymethyl cellulose, methyl cellulose, hydroxypropylmethyl cellulose, konjac, chitosan, tragacanth, karaya, ghatti, larch, carageenan, alginate, chemically modified alginate, agar, guar, locust bean, psyllium, tara, gellan, curdlan, pullan, gum arabic, gelatin, pectin, and combinations thereof.

75. The composition according to claim 66, wherein the edible substrate is selected from the group consisting of tea leaves, coffee beans, cocoa powder, meats, fish, fruits, vegetables, dairy products, legumes, pastas, breads, grains, seeds, nuts, spices, and herbs.

76. The composition according to claim 66, further comprising a flavoring agent selected from the group consisting of vanilla, vanillin, ethyl vanillin, orange oil, fruit and berry type flavorants, dramboui, bourbon, scotch, whiskey, spearmint, lavender, cinnamon, chai, cardamon, apium graveolents, clove, cascarilla, nutmeg, sandalwood, bergamot, geranium, honey essence, rose oil, lemon oil, Japanese mint, cassia, caraway, cognac, jasmin, chamomile, menthol, ylang ylang, sage, fennel, pimenta, ginger, anise, chai, coriander, coffee, peppermint, wintergreen, mint oils from a species of the genus Mentha, and combinations thereof.

77. The composition according to claim 66, further comprising an additive selected from the group consisting of a non-nicotine alkaloid, a mineral, a vitamin, a dietary supplement, a dietary mineral, a nutraceutical, an energizing agent, a soothing agent, a coloring agent, an amino acid, a chemesthetic agent, an antioxidant, a food grade emulsifier, a pH modifier, a botanical, a teeth whitening agent, a therapeutic agent, a sweetener, a flavorant, and combinations thereof.

78. A process for preparing an edible product infused with nicotine, a nicotine derivative, or a salt thereof, comprising:
a) providing a therapeutically effective amount of a nicotine compound;
b) providing a bioavailability enhancing agent, wherein the bioavailability agent comprises an edible oil;
c) providing an edible substrate;
d) contacting the edible substrate with the edible oil, nicotine compound and bioavailability enhancing agent to form an infused edible substrate; and
e) dehydrating the edible substrate.

79. The process according to claim 78, wherein the process comprises nicotine.

80. The process according to claim 78, comprising nicotine hydrogen tartrate, nicotine bitartrate dihydrate, nicotine hydrochloride, nicotine dihydrochloride, nicotine sulfate, nicotine citrate, nicotine zinc chloride monohydrate, nicotine salicylate, nicotine oil, or nicotine complexed with cyclodextrin.

81. The process according to claim 78, wherein the nicotine derivative is an agonist having selectivity to an α7 nicotinic receptor subtype, wherein the α7 nicotinic receptor subtype agonist is selected from the group consisting of N-[(2S,3S)-2-(pyridin-3-ylmethyl)-1-azabicyclo[2.2.2]oct-3-yl]-1-benzofuran-2-carboxamide, (5aS,8 S,10aR)-5a,6,9,10-Tetrahydro,7H,11H-8,10a-methanopyrido[2′,3′:5,6]pyrano[2,3-d]azepine, 1,4-diazabicyclo[3.2.2]nonane-4-carboxylic acid, 4-bromophenyl ester, 3-[(3E)-3-[(2,4-dimethoxyphenyl)methylidene]-5,6-dihydro-4H-pyridin-2-yl]-pyridine, 2-methyl-5-(6-phenyl-pyridazin-3-yl)-octahydro-pyrrolo[3,4-c]pyrrole, (5S)-spiro[1,3-oxazolidine-5,8′-1-azabicyclo[2.2.2]octane]-2-one, N-[(3R)-1-azabicyclo[2.2.2]oct-3-yl]-4-chlorobenzamide, 5-morpholin-4-yl-pentanoic acid (4-pyridin-3-yl-phenyl)-amide, EVP-6124, EVP-4473, TC-6987, and MEM3454, or the nicotine derivative is an agonist having selectivity to an α₄β₂ nicotinic receptor subtype, wherein the α₄β₂ nicotinic receptor subtype agonist is selected from the group consisting of 7,8,9,10-tetrahydro-6,10-methano-6H-pyrazino(2,3-h)(3) benzazepine, (2 S,4E)-5-(5-isopropoxypyridin-3-yl)-N-methylpent-4-en-2-amine, [3-(2(S))-azetidinylmethoxy)pyridine] dihydrochloride, (5aS,8S,10aR)-5a,6,9,10-Tetrahydro,7H,11H-8,10a-methanopyrido [2′,3′:5,6]pyrano[2,3-d]azepine, A-969933, S35836-1, S35678-1, and 3-(5,6-Dichloro-pyridin-3-yl)-1S,5S-3,6-diazabicyclo[3.2.0]heptane.

82. The process according to claim 78, wherein the bioavailability enhancing agent bioavailability enhancing agent is a protective colloid, an edible oil or fat.

83. The process according to claim 82, wherein the edible oil is coconut oil, peanut oil, soybean oil, safflower seed oil, corn oil, olive oil, castor oil, cottonseed oil, arachis oil, or sunflower seed oil.

84. The process according to claim 83, wherein the edible oil comprises fatty acids selected from the group consisting of oleic acid, undecanoic acid, valeric acid, heptanoic acid, pelargonic acid, capric acid, lauric acid, and eicosapentaenoic acid.

85. The process according to claim 78, wherein the edible substrate is selected from the group consisting of inulin, starch, modified starches, xanthan gum, carboxymethyl cellulose, methyl cellulose, hydroxypropylmethyl cellulose, konjac, chitosan, tragacanth, karaya, ghatti, larch, carageenan, alginate, chemically modified alginate, agar, guar, locust bean, psyllium, tara, gellan, curdlan, pullan, gum arabic, gelatin, pectin, and combinations thereof.

86. The process according to claim 78, wherein the edible substrate is selected from the group consisting of tea leaves, coffee beans, cocoa powder, meats, fish, fruits, vegetables, dairy products, legumes, pastas, breads, grains, seeds, nuts, spices, and herbs.

87. A process for preparing a beverage product infused with nicotine, a nicotine derivative, or a salt thereof, obtainable by the steps of:
a) providing a composition according to claim 66 wherein the edible substrate is tea leaves, coffee beans, or cocoa powder;
b) dehydrating the composition; and
c) steeping the composition in a liquid;
thereby producing the beverage product infused with the nicotine compound.

88. The process according to claim 87, wherein the beverage product comprises nicotine.

89. The process according to claim 87, comprising nicotine hydrogen tartrate, nicotine bitartrate dihydrate, nicotine hydrochloride, nicotine dihydrochloride, nicotine sulfate, nicotine citrate, nicotine zinc chloride monohydrate, nicotine salicylate, nicotine oil, or nicotine complexed with cyclodextrin.

90. The process according to claim 87, wherein the nicotine derivative is an agonist having selectivity to an α₇ nicotinic receptor subtype, wherein the α₇ nicotinic receptor subtype agonist is selected from the group consisting of N-[(2S,3S)-2-(pyridin-3-ylmethyl)-1-azabicyclo[2.2.2]oct-3-yl]-1-benzofuran-2-carboxamide, (5aS,8S,10aR)-5a,6,9,10-Tetrahydro,7H,11H-8,10a-methanopyrido[2′,3′:5,6]pyrano[2,3-d]azepine, 1,4-diazabicyclo[3.2.2]nonane-4-carboxylic acid, 4-bromophenyl ester, 3-[(3E)-3-[(2,4-dimethoxyphenyl)methylidene]-5,6-dihydro-4H-pyridin-2-yl]-pyridine, 2-methyl-5-(6-phenyl-pyridazin-3-yl)-octahydro-pyrrolo[3,4-c]pyrrole, (5S)-spiro[1,3-oxazolidine-5,8′-1-azabicyclo[2.2.2]octane]-2-one, N-[(3R)-1-azabicyclo[2.2.2]oct-3-yl]-4-chlorobenzamide, 5-morpholin-4-yl-pentanoic acid (4-pyridin-3-yl-phenyl)-amide, EVP-6124, EVP-4473, TC-6987, and MEM3454, or the nicotine derivative is an agonist having selectivity to an α₄β₂ nicotinic receptor subtype, wherein the α₄β₂ nicotinic receptor subtype agonist is selected from the group consisting of 7,8,9,10-tetrahydro-6,10-methano-6H-pyrazino(2,3-h)(3) benzazepine, (2S,4E)-5-(5-isopropoxypyridin-3-yl)-N-methylpent-4-en-2-amine, [3-(2(S))-azetidinylmethoxy)pyridine] dihydrochloride, (5aS,8S,10aR)-5a,6,9,10-Tetrahydro,7H,11H-8,10a-methanopyrido [2′,3′:5,6]pyrano[2,3-d]azepine, A-969933, S35836-1, S35678-1, and 3-(5,6-Dichloro-pyridin-3-yl)-1S,5S-3,6-diazabicyclo[3.2.0]heptane.

91. The process according to claim 87, wherein the bioavailability enhancing agent bioavailability enhancing agent is a protective colloid, an edible oil or fat.

92. The process according to claim 91, wherein the edible oil is coconut oil, peanut oil, soybean oil, safflower seed oil, corn oil, olive oil, castor oil, cottonseed oil, arachis oil, or sunflower seed oil.

93. The composition according to claim 92, wherein the edible oil comprises fatty acids selected from the group consisting of oleic acid, undecanoic acid, valeric acid, heptanoic acid, pelargonic acid, capric acid, lauric acid, and eicosapentaenoic acid.

94. The composition according to claim 87, further comprising a flavoring agent selected from the group consisting of vanilla, vanillin, ethyl vanillin, orange oil, fruit and berry type flavorants, Dramboui, bourbon, scotch, whiskey, spearmint, lavender, cinnamon, chai, cardamon, apium graveolents, clove, cascarilla, nutmeg, sandalwood, bergamot, geranium, honey essence, rose oil, lemon oil, Japanese mint, cassia, caraway, cognac, jasmin, chamomile, menthol, ylang ylang, sage, fennel, pimenta, ginger, anise, chai, coriander, coffee, peppermint, wintergreen, mint oils from a species of the genus Mentha, and combinations thereof.

95. The composition according to claim 87, further comprising an additive selected from the group consisting of a non-nicotine alkaloid, a mineral, a vitamin, a dietary supplement, a dietary mineral, a nutraceutical, an energizing agent, a soothing agent, a coloring agent, an amino acid, a chemesthetic agent, an antioxidant, a food grade emulsifier, a pH modifier, a botanical, a teeth whitening agent, a therapeutic agent, a sweetener, a flavorant, and combinations thereof.
